SEQUENCE LISTING (14 sequences)

SEQ ID NO: 1 - DNA, 100 bp, synthetic construct
  source 1..100: mol_type = other DNA
tttttttttttttgatgatgtgaaggtgttgtcgctaactaactaactaactaactaactaactaactaactaactactatctttcctcttaattcgacg

SEQ ID NO: 2 - DNA, 120 bp, synthetic construct
  source 1..120: mol_type = other DNA
tttttttttttttgatgatgtgaaggtgttgtcgctaactaactaactaactaactaactaactaactaactaactaactaactaactaactaactactatctttcctcttaattcgacg

SEQ ID NO: 3 - DNA, 140 bp, synthetic construct
  source 1..140: mol_type = other DNA
tttttttttttttgatgatgtgaaggtgttgtcgctaactaactaactaactaactaactaactaactaactaactaactaactaactaactaactaactaactaactaactaactactatctttcctcttaattcgacg

SEQ ID NO: 4 - DNA, 160 bp, synthetic construct
  source 1..160: mol_type = other DNA
tttttttttttttgatgatgtgaaggtgttgtcgctaactaactaactaactaactaactaactaactaactaactaactaactaactaactaactaactaactaactaactaactaactaactaactaactaactactatctttcctcttaattcgacg

SEQ ID NO: 5 - DNA, 200 bp, synthetic construct
  source 1..200: mol_type = other DNA
tttttttttttttgatgatgtgaaggtgttgtcgctaactaactaactaactaactaactaactaactaactaactaactaactaactaactaactaactaactaactaactaactaactaactaactaactaactaactaactaactaactaactaactaactaactaactaactactatctttcctcttaattcgacg

SEQ ID NO: 6 - DNA, 23 bp, synthetic construct
  source 1..23: mol_type = other DNA
cgtcgaattaagaggaaagatag

SEQ ID NO: 7 - DNA, 20 bp, synthetic construct
  source 1..20: mol_type = other DNA
tactacacttccacaacagc

SEQ ID NO: 8 - RNA, 41 nt, synthetic construct
  source 1..41: mol_type = other RNA
  ncRNA 1..41: ncRNA_class = guide_RNA
  modified_base 1: mod_base = OTHER; note = uracil
  modified_base 8: mod_base = OTHER; note = uracil
  modified_base 6: mod_base = OTHER; note = uracil
  modified_base 17: mod_base = OTHER; note = uracil
  modified_base 15: mod_base = OTHER; note = uracil
  modified_base 37: mod_base = OTHER; note = uracil
  modified_base 5: mod_base = OTHER; note = uracil
  modified_base 23: mod_base = OTHER; note = uracil
  modified_base 4: mod_base = OTHER; note = uracil
  modified_base 28: mod_base = OTHER; note = uracil
  modified_base 34: mod_base = OTHER; note = uracil
  modified_base 39: mod_base = OTHER; note = uracil
  modified_base 11: mod_base = OTHER; note = uracil
  modified_base 26: mod_base = OTHER; note = uracil
  modified_base 21: mod_base = OTHER; note = uracil
  modified_base 36: mod_base = OTHER; note = uracil
taatttctactaagtgtagatatgatgtgaaggtgttgtcg

SEQ ID NO: 9 - DNA, 200 bp, synthetic construct
  source 1..200: mol_type = other DNA
tttttttttttttgatgatgtgaaggtgttgtcgttcattcattcattcattcattcattcattcattcattcattcattcattcattcattcattcattcattcattcattcattcattcattcattcattcattcattcattcattcattcattcattcattcattcattcattcctatctttcctcttaattcgacg

SEQ ID NO: 10 - DNA, 80 bp, synthetic construct
  source 1..80: mol_type = other DNA
tttttttttttttgatgatgtgaaggtgttgtcgttcattcattcattcattcattcctatctttcctcttaattcgacg

SEQ ID NO: 11 - DNA, 200 bp, synthetic construct
  source 1..200: mol_type = other DNA
tttttttttttttgatgatgtgaaggtgttgtcgtttatttatttatttatttatttatttatttatttatttatttatttatttatttatttatttatttatttatttatttatttatttatttatttatttatttatttatttatttatttatttatttatttatttatttatttctatctttcctcttaattcgacg

SEQ ID NO: 12 - DNA, 80 bp, synthetic construct
  source 1..80: mol_type = other DNA
tttttttttttttgatgatgtgaaggtgttgtcgtttatttatttatttatttatttctatctttcctcttaattcgacg

SEQ ID NO: 13 - DNA, 120 bp, synthetic construct
  source 1..120: mol_type = other DNA
tttttttttttttgatgatgtgaaggtgttgtcgtttatttatttatttatttatttatttatttatttatttatttatttatttatttatttatttctatctttcctcttaattcgacg

SEQ ID NO: 14 - DNA, 140 bp, synthetic construct
  source 1..140: mol_type = other DNA
tttttttttttttgatgatgtgaaggtgttgtcgtttatttatttatttatttatttatttatttatttatttatttatttatttatttatttatttatttatttatttatttatttctatctttcctcttaattcgacg

(19) United States
(12) Patent Application Publication
(10) Pub. No.: US 2026/0139294 A1
(43) Pub. Date: May 21, 2026

(54) DEVELOPMENT OF A RAPID ENZYMATIC ASSAY FOR ANTIRETROVIRAL DRUG MONITORING USING CRISPR REPORTERS

(71) Applicant: University of Washington, Seattle, WA (US)

(72) Inventors: Maya Singh, Seattle, WA (US); Ayokunle Olanrewaju, Seattle, WA (US); Megan Chang, Seattle, WA (US); Qin Wang, Seattle, WA (US); Catherine Rodgers, Seattle, WA (US); Barry Ryan Lutz, Seattle, WA (US); Carrie HsuanYu Lin, Seattle, WA (US)

(73) Assignee: University of Washington, Seattle, WA (US)

(21) Appl. No.: 19/396,892

(22) Filed: Nov. 21, 2025

Related U.S. Application Data
(60) Provisional application No. 63/723,495, filed on Nov. 21, 2024.

(51) Int. Cl. C12Q 1/6823; C12N 15/11; C12Q 1/34; C12Q 1/70; G01N 21/64; G01N 33/543

(52) U.S. Cl. CPC C12Q 1/6823; C12N 15/11; C12Q 1/34; C12Q 1/703; G01N 21/6428; G01N 33/54388; C12N 2310/20; C12Q 2600/106; G01N 2021/6432; G01N 2333/9128; G01N 2333/922

(57) ABSTRACT

Provided herein are systems and methods for detecting a target enzyme and/or target enzyme activity. In some embodiments, the system comprises: (a) a CRISPR effector system; and (b) a nucleic acid probe comprising a detectable label. In some embodiments, the CRISPR effector system comprises: (i) at least one CRISPR effector protein having collateral cleavage activity; and (ii) at least one sgRNA that directs the at least one CRISPR effector protein to specifically bind to a target nucleic acid sequence. In some embodiments, the target nucleic acid sequence is a sequence generated/synthesized by the activity of the target enzyme. In some embodiments, the nucleic acid probe is cleavable by collateral cleavage activity of the at least one CRISPR effector protein. Also provided herein is a point of care method for monitoring adherence to a therapy targeting a disease-associated enzyme in a subject using the systems disclosed herein.

### CROSS REFERENCE TO RELATED APPLICATION

This application claims the benefit of U.S. Provisional Application No. 63/723,495, filed Nov. 21, 2024, the disclosure of which is incorporated herein by reference in its entirety.

### STATEMENT OF GOVERNMENT LICENSE RIGHTS

This invention was made with Government support under R33AI140460 awarded by National Institute of Allergy & Infectious Diseases (NIAID). The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3915-P1372USUW_Seq_List_20251119.xml. The XML file is 23,608 bytes; was created on Nov. 19, 2025; and is being submitted electronically via Patent Center with the filing of the specification.

### BACKGROUND

Human Immunodeficiency Virus (HIV) infection remains a significant global health concern, including among pediatric populations. Antiretroviral therapy (ART) can effectively suppress viral replication when taken as prescribed. However, treatment outcomes are highly dependent on adherence, and maintaining consistent adherence in pediatric patients presents ongoing challenges.

Pediatric ART dosing often requires weight-based adjustments and relies on caregivers for administration. Multiple daily doses, poor palatability, and caregiver or patient fatigue can contribute to missed or delayed doses. Limited follow-up and social factors may further affect adherence consistency.

Current methods for monitoring adherence, including self-reporting, pill counts, pharmacy refill tracking, and electronic medication event monitoring systems, are indirect and subject to reporting bias or behavioral inaccuracies. These approaches may not reliably confirm ingestion of medication or provide timely feedback to clinicians.

Biological monitoring of drug levels can offer objective adherence data but typically involves invasive sampling, laboratory-based assays, and delayed result availability. Existing point-of-care and digital adherence tools often operate independently of drug level assessment and may not provide integrated, real-time feedback suitable for pediatric use.

Accordingly, there is a need for improved systems and methods for monitoring adherence to antiretroviral therapy in pediatric HIV patients. In particular, there is a need for approaches that provide objective drug level feedback (DLF), are low-cost, and provide point-of-care assessment capable of providing DLF for antiretroviral therapeutic agents to facilitate monitoring of patient adherence to prescribed therapies and/or enhancing compliance with medication regimens, which may be integrated with existing monitoring platforms to enable timely and effective adherence management.

### SUMMARY

The present disclosure provides systems and methods for monitoring adherence to antiretroviral therapy in pediatric patients. The systems and methods described are also broadly applicable to other populations that require HIV medication adherence. In certain embodiments, the systems and methods integrate objective drug level feedback at point-of-care to digital adherence monitoring platforms to provide near real-time or periodic assessments of medication intake. Particularly, the present disclosure provides a rapid point-of-care, and adaptable systems, devices, and methods for monitoring drug adherence, specifically ART adherence in pediatric patients suffering from HIV, thereby improving the management of HIV in children.

In some embodiments, the present disclosure provides a system for detecting a target enzyme, the system comprising (a) a CRISPR effector system comprising (i) at least one CRISPR effector protein having collateral cleavage activity; and (ii) at least one sgRNA that directs the at least one CRISPR effector protein to specifically bind to a target nucleic acid sequence; and (b) a detection system comprising a nucleic acid probe comprising a detectable label. In certain embodiments, the target nucleic acid sequence is a sequence generated/synthesized by the activity of the target enzyme. In some embodiments, the nucleic acid probe is cleavable by collateral cleavage activity of at least one CRISPR effector protein upon activation. In an embodiment, when the nucleic acid probe is cleaved a detectable signal is generated from the detectable label or when the nucleic acid probe is cleaved a detectable signal is removed from the detectable label.

In some embodiments, the system further comprises reagents for generation of the target nucleic acid, and wherein the reagents comprise: (i) a nucleic acid template; (ii) dNTPs; and (iii) primers. In an embodiment, at least one CRISPR effector protein comprises one or more thermostable Cas proteins possessing collateral activity, and wherein one or more Cas protein is a Type V Cas. In some embodiments, at least one CRISPR effector protein comprises a Cas 12a protein or a protein with similar bypass/collateral cleavage activity as Cas12a. In some embodiments, at least one CRISPR effector protein sgRNA is a crRNA molecule.

In an embodiment, when the target enzyme is not active or is absent, the target nucleic acid sequence is not generated, the CRISPR effector system is inactive, and the nucleic acid probe is not cleaved by the collateral cleavage activity of the at least one CRISPR effector protein to generate the detectable signal from the detectable label or to remove the detectable signal from the detectable label.

In another embodiment, when the target enzyme is active or present, the target nucleic acid sequence is generated, the CRISPR effector system is active, and the nucleic acid probe is cleaved by the collateral cleavage activity of the at least one CRISPR effector protein to generate the detectable signal or to remove the detectable signal from the detectable label.

In some embodiments, the CRISPR-effector system is activated by a target enzyme-responsive nucleic acid signal that correlates with the presence or concentration of the drug. In some embodiments, the drug or the therapeutic agent inhibits the activity or the expression of the target enzyme. In some embodiments, the target enzyme-responsive nucleic acid signal comprises generation of the target nucleic acid by the target enzyme. In some embodiments, the target enzyme-responsive nucleic acid signal comprises non-generation of the target nucleic acid by the target enzyme.

In presence of the drug, the target enzyme activity is inhibited, the target nucleic acid is not generated, and the CRISPR effector system is inactive. In absence or at low concentrations of the drug, the target enzyme activity is not inhibited, the target nucleic acid is generated, and the CRISPR effector system is active.

In some embodiments, the generation or removal of the detectable signal is detected using a detection equipment/instrument. In some embodiments, the generation or removal of the detectable signal is visually detected by naked eyes without an instrument/detection equipment. In an embodiment, the nucleic acid probe comprises a first molecule attached to the 5′ end and a second molecule attached to the 3′ ends of an oligo or vice-versa, or the nucleic acid probe comprises a first molecule attached on any base or multiple bases of the oligo and a second molecule attached to a different base of the oligo.

In some embodiments, the nucleic acid probe is an RNA or DNA oligonucleotide. In certain embodiments, the nucleic acid probe is a single-stranded RNA or a single-stranded DNA oligonucleotide, and wherein the first molecule and the second molecule are covalently attached on the base of the single-stranded RNA or single-stranded DNA oligonucleotide. In an embodiment, the first molecule is a fluorescent molecule selected from FITC, Cy5, FAM, Texas Red, and Cy7. In some embodiments, the second molecule is a fluorescent quencher, wherein the fluorescent quencher is selected from BHQ-1, BHQ-2, Dabcyl, and Iowa Black FQ. In some embodiments, the system is present in a lateral flow format.

In an embodiment, the target enzyme is a pathogen-derived enzyme. In some embodiments, the system detects the activity of the pathogen-derived enzyme following exposure to an anti-pathogenic agent. In some embodiments, the pathogen-derived enzyme is a reverse transcriptase enzyme or a retroviral integrase of the Human immunodeficiency virus (HIV), and the anti-pathogenic agent is an Antiretroviral therapeutic (ART) agent. In an embodiment, the system is used for monitoring levels of the anti-pathogenic agent based on activity of the target enzyme.

In another aspect, the present disclosure provides a visual detection system for detecting/monitoring enzymatic activity of a target enzyme, the system comprising (a) a CRISPR effector system comprising (i) at least one CRISPR effector protein, said CRISPR effector protein being Cas12a or a Cas protein with similar collateral cleavage activity as Cas12a; and (ii) at least one sgRNA that directs the at least one CRISPR effector protein to specifically bind to a target nucleic acid sequence; and (b) a nucleic acid probe comprising a visually detectable label. In certain embodiments, the target nucleic acid sequence is a sequence generated/synthesized by the activity of the target enzyme. In some embodiments, the nucleic acid probe is cleavable by collateral cleavage activity of the at least one CRISPR effector protein upon activation. In an embodiment, when the nucleic acid probe is cleaved a detectable signal is generated from the detectable label or when the nucleic acid probe is cleaved a detectable signal is removed from the detectable label.

In some embodiments, the system further comprises reagents for generation of the target nucleic acid, and wherein the reagents comprise: (i) a nucleic acid template; (ii) dNTPs; and (iii) primers. In an embodiment, at least one CRISPR effector protein comprises one or more thermostable Cas proteins possessing collateral activity. In some embodiments, the one or more Cas protein is a Type V Cas. In some embodiments, at least one CRISPR effector protein comprises a Cas 12a protein or a protein with similar bypass/collateral cleavage activity as Cas12a. In some embodiments, at least one CRISPR effector protein sgRNA is a crRNA molecule.

In an embodiment, when the target enzyme is not active or is absent, the target nucleic acid sequence is not generated, the CRISPR effector system is inactive, and the nucleic acid probe is not cleaved by the collateral cleavage activity of the at least one CRISPR effector protein to generate the detectable signal from the detectable label or to remove the detectable signal from the detectable label.

In another embodiment, when the target enzyme is active or present, the target nucleic acid sequence is generated, the CRISPR effector system is active, and the nucleic acid probe is cleaved by the collateral cleavage activity of the at least one CRISPR effector protein to generate the detectable signal or to remove the detectable signal from the detectable label.

In some embodiments, the CRISPR-effector system is activated by a target enzyme-responsive nucleic acid signal that correlates with the presence or concentration of the drug. In some embodiments, the drug or the therapeutic agent inhibits the activity or the expression of the target enzyme. In some embodiments, the target enzyme-responsive nucleic acid signal comprises generation of the target nucleic acid by the target enzyme. In some embodiments, the target enzyme-responsive nucleic acid signal comprises non-generation of the target nucleic acid by the target enzyme.

In presence of the drug, the target enzyme activity is inhibited, the target nucleic acid is not generated, and the CRISPR effector system is inactive. In absence or at low concentrations of the drug, the target enzyme activity is not inhibited, the target nucleic acid is generated, and the CRISPR effector system is active.

In some embodiments, the visually detectable signal is a fluorescence signal detectable by naked eye or by means of fluorescence detection equipment or wherein the visually detectable signal is a color change detectable directly by naked eye. In some embodiments, the nucleic acid probe comprises a first molecule attached to the 5′ end and a second molecule attached to the 3′ ends of an oligo or vice-versa, or wherein the nucleic acid probe comprises a first molecule attached on any base of an oligo or multiple bases of an oligo and a second molecule attached to a different base of the oligo.

In an embodiment, the nucleic acid probe is an RNA or DNA oligonucleotide. In some embodiments, the nucleic acid probe is a single-stranded RNA or a single-stranded DNA oligo, and the first molecule and the second molecule are covalently attached on a base of the oligo. In some embodiments, the first molecule is a fluorescent molecule selected from FITC, Cy5, FAM, Texas Red, and Cy7. In an embodiment, the second molecule is a fluorescent quencher, wherein the fluorescent quencher is selected from BHQ-1, BHQ-2, Dabcyl, and Iowa Black FQ.

In some embodiments, the present disclosure provides a lateral flow device/system for detecting/monitoring enzymatic activity of a target enzyme, the device/system comprising a substrate comprising a first end. In some embodiments, the first end comprises (a) a sample loading portion; and (b) a first region. In some embodiments, the first region comprises (i) a CRISPR effector system; (ii) a nucleic acid probe comprising a detectable label; and (iii) a first capture region comprising a first binding agent. In an embodiment, the CRISPR effector system comprises at least one CRISPR effector protein having collateral cleavage activity; and at least one sgRNA that directs at least one CRISPR effector protein to specifically bind to a target nucleic acid sequence. In some embodiments, the target nucleic acid sequence is a sequence generated/synthesized by the enzymatic activity of the target enzyme, wherein the nucleic acid probe is cleavable by collateral cleavage activity of the CRISPR effector system, and wherein when the nucleic acid probe is cleaved a detectable signal is generated from the detectable label or wherein when the nucleic acid probe is cleaved a detectable signal is removed from the detectable label.

In some embodiments, the first capture region is proximate to and on the same end of the lateral flow substrate as the sample loading portion. In an embodiment, the first capture region comprises a first binding agent that specifically binds the first molecule of the nucleic acid probe.

In some embodiments, the lateral flow device/system further comprises a second capture region. In some embodiments, the second capture region is located towards the opposite end of the lateral flow substrate from the first binding region. In an embodiment, the second capture region optionally comprises a second binding agent that specifically binds the second molecule of the nucleic acid probe. In an embodiment, the first and the second capture regions are in fluid communication.

In some embodiments, the sample loading portion is configured to collect a biological sample, such as a small-volume blood, saliva, or other bodily fluid sample, suitable for determining drug concentration. The system may further include a drug level detection module configured to measure one or more antiretroviral drug levels using analytical, biosensor, or immunoassay-based detection techniques. The results of the measurement may be processed to generate adherence data correlated with prescribed dosing regimens.

In certain embodiments, the adherence data are transmitted to or integrated with an electronic monitoring platform or digital health interface. The platform may provide feedback to clinicians, caregivers, or patients, and may include features such as adherence alerts, trend analyses, and data visualization tools. Integration with existing adherence monitoring systems may enable consolidated tracking of behavioral and pharmacologic adherence indicators.

In some embodiments, the sample is a biological sample; wherein the biological sample is optionally a blood, plasma, serum, urine, saliva, mucous, cervicovaginal fluid, lymph fluid, synovial fluid, ascites, pleural effusion, seroma, cerebrospinal fluid, sputum, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

In some embodiments, the sample is loaded in the sample loading portion of the substrate. In an embodiment, the sample flows from the sample loading portion of the substrate to the first and optionally the second capture regions. In some embodiments, when the target enzyme is not active or is absent in the sample, the target nucleic acid sequence is not generated, the CRISPR effector system is inactive, and the nucleic acid probe is not cleaved by the collateral cleavage activity of the at least one CRISPR effector protein to generate a visually detectable signal from the detectable label or to remove a visually detectable signal from the detectable label. In an embodiment, when the target enzyme is active or present, the target nucleic acid sequence is generated, the CRISPR effector system is active, and the nucleic acid probe is cleaved by collateral cleavage activity of the CRISPR effector protein to generate a visually detectable signal from the detectable label or to remove a visually detectable signal from the detectable label.

In still yet another embodiment, the present disclosure provides a system for monitoring a drug level in a biological sample. In some embodiments, the system comprises (a) a lateral flow assay device comprising a sample pad, a conjugate pad, a nitrocellulose membrane, and an absorbent pad arranged in fluid communication; (b) a CRISPR/Cas detection complex disposed on or within the conjugate pad, wherein the CRISPR/Cas detection complex comprises a guide RNA and a Cas effector enzyme configured to cleave a labeled nucleic acid reporter in response to a drug-responsive nucleic acid signal: (c) a nucleic acid probe or a reporter configured to produce a detectable signal upon cleavage; and (d) a digital reader configured to detect the signal and generate an output indicative of the concentration of the drug in the biological sample.

In some embodiments, the drug-responsive nucleic acid signal correlates with the presence or concentration of the drug. In some embodiments, the drug modulates the activity or the expression of a target enzyme. In some embodiments, the target enzyme generates the drug-responsive nucleic acid signal. In some embodiments, the drug-responsive nucleic acid comprises generation or non-generation of a target nucleic acid site. In some embodiments, the activity of the CRISPR/Cas detection complex is modulated by the generation or non-generation of the target nucleic acid site.

In some embodiments, in presence of the drug, the target enzyme activity is inhibited, the target nucleic acid is not generated, and the CRISPR/Cas detection complex is inactive. In some embodiments, the drug-responsive nucleic acid signal comprises absence or low concentrations of the drug. In absence or at low concentrations of the drug, the target enzyme activity is not inhibited, the target nucleic acid is generated and the CRISPR effector system is active.

In some embodiments, the Cas effector enzyme is selected from the group consisting of Cas12a (Cpf1), Cas13a, Cas13b, Cas14a (Cas12f), and CasDx1. In an embodiment, the drug-responsive nucleic acid signal comprises absence or low concentrations of the drug level in the biological sample. In an embodiment, the detectable signal is colorimetric, fluorescent, chemiluminescent, or electrochemical. In some embodiments, the reporter comprises a fluorophore-quencher pair, a biotin-fluorescein pair, or a nanoparticle conjugate. In an embodiment, the digital reader comprises an optical sensor configured to detect reflected light, fluorescence intensity, or luminescence from a test line and a control line on the lateral flow strip. In some embodiments, the digital reader includes a processor configured to normalize and analyze the signal intensity using a calibration curve or algorithm to compute a quantitative drug concentration.

In an embodiment, the present disclosure provides a system for integrated drug adherence monitoring comprising (a) a CRISPR-based lateral flow assay configured to detect a drug or metabolite in a biological sample; (b) a digital reader configured to quantify the assay signal; and (c) a software platform configured to process and store temporal drug level data, compare measured concentrations to expected pharmacokinetic profiles, and provide adherence feedback or dosing recommendations.

In yet another embodiment, the disclosure provides a point of care method for monitoring adherence to a therapy targeting a disease-associated enzyme in a subject. In an embodiment, the method comprises (i) obtaining a biological sample from the subject; (ii) contacting the biological sample with the systems disclosed herein; (iii) measuring the detectable signal generated or removed from the detectable label; and (iv) determining a level of adherence to the therapy based on the measured detectable signal. In some embodiments, the disease-associated enzyme is reverse transcriptase of Human immunodeficiency virus (HIV). In some embodiments, the disease-associated enzyme is an integrase of Human immunodeficiency virus (HIV). In an embodiment, the therapy comprises an antiretroviral therapy (ART). In some embodiments, the ART comprises tenofovir-diphosphate (TFV-DP).

In yet another embodiment, the disclosure provides a method for determining a drug concentration in a biological sample, comprising introducing the biological sample to a lateral flow assay disclosed herein. In some embodiments, the lateral flow assay comprises a CRISPR/Cas detection complex and a labeled reporter. In some embodiments, the method further comprises activating the CRISPR/Cas detection complex via a drug-responsive nucleic acid signal that correlates with the presence or concentration of the drug; detecting a cleavage event of the reporter to generate a measurable signal; and processing the measurable signal using a digital reader to determine or estimate the drug concentration and optionally transmitting the result to a remote or local data management platform.

The disclosed systems and methods may allow objective, minimally invasive, and user-friendly assessment of drug adherence in pediatric HIV patients. By combining drug level measurement with digital feedback, the technology can facilitate early detection of non-adherence, support individualized intervention, and improve overall treatment management.

Other objects, features, and advantages of the present invention will be apparent to one of ordinary skill in the art from the following detailed description and drawings.

### BRIEF DESCRIPTION OF DRAWINGS

FIGS. 1A-1B provide an overview of REACTR assay. The REACTR assay measures the concentration of nucleotide reverse transcriptase inhibitors (NRTIs) as a function of DNA synthesis by HIV reverse transcriptase (RT). At high NRTI levels. DNA chain termination occurs and inactivated Cas12a complexes are unable to generate fluorescence. However, at low NRTI levels, Cas12a complexes are activated and cleave reporter complexes leading to increased fluorescence (FIG. 1A). An example schematic of real-time fluorescence curves obtained with REACTR at low, medium, and high NRTI concentrations (FIG. 1B).

FIGS. 2A-2C shows REACTR real-time performance and characterization with AZT-TP. Real-time REACTR curves with varying concentrations of AZT-TP. Fluorescence measurements were collected every 2 minutes. Each data point represents the mean fluorescence measurement of three replicates (FIG. 2A). Inhibition curves were generated from (FIG. 2B) raw fluorescence and (FIG. 2C) normalized fluorescence as a function of AZT-TP concentrations at different time points (N=3).

FIGS. 3A-3D show impact of DNA Template Length on REACTR. FIG. 3A is a diagram of the DNA template used for REACTR experiments. The template includes an RT primer binding region followed by CTAA repeats, ranging from 5 to 35 iterations, resulting in templates 80 to 200 nucleotides in length. The repeats are followed by the complement to the crRNA recognition sequence, a PAM terminating sequence, and a Poly T-tail. Raw fluorescence data (FIG. 3B) and normalized fluorescence data (FIG. 3C) plotted for varying template lengths for AZT-TP concentrations (N=3). Data was analyzed at the 30-minute time point. FIG. 3D shows calculated IC50 values as a function of DNA template length. Error bars indicate 95% confidence intervals. Grey bands indicate clinical range of AZT-TP that accumulates in white blood cells.

FIGS. 4A-4C show REACTR in the presence of genomic DNA. FIG. 4A shows positive and negative controls at different genomic DNA concentrations. A solid line for each condition represents the mean (N=3). FIG. 4B shows fluorescence drug inhibition curves for AZT-TP concentrations for reactions containing no genomic DNA concentrations. 4 ng/L of genomic DNA, and 10 ng/μL of genomic DNA (N=3). FIG. 4C shows normalized drug inhibition curves.

FIGS. 5A-5C show REACTR performance in PBMC lysate. FIG. 5A shows positive and negative controls for different PBMC lysate dilutions ranging from 4% to 100% (N=3). The red and blue dotted lines represent the negative and positive buffer controls, respectively, with TE buffer. FIG. 5B shows fluorescence inhibition curves for varying AZT-TP concentrations with N=3. Two PBMC lysate concentrations. 5E5 cells/mL and 2E6 cells/mL, were chosen and compared to a no PBMC lysate control. Data were analyzed 30 minutes after incubation. FIG. 5C shows normalized drug inhibition curves.

FIG. 6 shows REACTR real-time performance and characterization curve with varying concentrations of AZT-TP data set in triplicate with error bars. Each data point represents the mean fluorescence measurements with error bars representing standard deviation (N=3).

FIGS. 7A-7D show REACTR real-time performance and characterization for each CTAA Template. FIG. 7A shows real-time REACTR curves with varying concentrations of AZT-TP. Each data point represents the mean fluorescence measurements (N=3) for 80 nt template (FIG. 7A). 100 nt template (FIG. 7B). 120 nt template (FIG. 7C), and 160 nt template (FIG. 7D).

FIG. 8 shows the addition of Genomic DNA to Intercalating Dye Based REACTR. PicoGreen was used as the intercalating dye to measure the fluorescence intensity after a 30-minute incubation period for the 200nt CTAA template. The addition of gDNA caused an increase in fluorescence and had indistinguishable signal between AZT-TP concentrations. Distinguishable fluorescence values were observed for each AZT-TP concentration in the PicoGreen only condition.

FIGS. 9A-9B show REACTR real-time performance curves for varying genomic DNA concentrations. Real-time REACTR curves spiked with 4 ng/μL genomic DNA with varying concentrations of AZT-TP. Each data point represents the mean fluorescence measurements (N=3) (FIG. 9A). Real-time REACTR curves spiked with 10 ng/μL genomic DNA with varying concentrations of AZT-TP. Each data point represents the mean fluorescence measurements (N=3) (FIG. 9B).

FIG. 10 shows varying PBMC lysate concentrations with PicoGreen readout. Concentrations of PBMC lysate starting from 2E6 cells/mL were diluted with TE Buffer. Since PBMC lysate has double stranded DNA, a decrease in fluorescence as the PBMC lysate concentration is decreased was expected. The fluorescence intensity was compared with a TE buffer only condition. PBMC lysate was incubated with PicoGreen at 37° C. for one minute before fluorescence measurements were taken.

FIGS. 11A-11B show REACTR real-time performance curves for varying PBMC lysate concentrations. Real-time REACTR curves spiked with 2E6 cells/mL PBMC lysate with varying concentrations of AZT-TP (FIG. 11A). Real-time REACTR curves spiked with 5E5 cells/mL PBMC lysate genomic DNA with varying concentrations of AZT-TP. Each data point represents the mean fluorescence measurements (N=3) (FIG. 11B).

FIG. 12 shows comparison of adherence testing mechanisms.

FIG. 13 shows the REACTR enzymatic assay overview using a lateral flow (LFA) readout. At high NRTI levels. DNA chain termination occurs and inactivated Cas12a complexes are unable to cleave the reporter leading to capture on the test line. However, at low NRTI levels, Cas12a complexes are activated and cleave reporter complexes remain uncaptured, and a test line does not develop.

FIG. 14 shows correlation between LC-MS/MS measurements and LFA signal with medium (700 fmol/punch) adherence to TFV-DP. 100% sensitivity and 100% specificity from 30 people taking TFV-DP based PrEP around 350 fmol/punch. 700 fmol/punch, and 1250 fmol/punch thresholds.

FIG. 15 shows scanned LFA strips and quantified intensity of quantified test line intensities using signal to background ratio (SBR).

### DETAILED DESCRIPTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided. The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosure. It is to be understood that the value to which the modifier “about” or “approximately” refers is itself also specifically, and preferably, disclosed.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

Throughout this disclosure, various aspects are presented in a range format. The description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed: more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A. B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising.” otherwise analogous aspects described in terms of “consisting of” and/or “consisting essentially of” are also provided.

“Oligonucleotide,” “polynucleotide,” and “nucleic acid,” are used interchangeably herein. These terms may refer to a polymeric form of nucleic acids of any length, strandedness (double or single), and either ribonucleotides (RNA) or deoxyribonucleotides (DNA), and hybrid molecules (comprising DNA and RNA). The disclosed nucleic acids may also include naturally occurring and synthetic or non-natural nucleobases. Natural nucleobases include adenine (A), thymine (T), cytosine (C), guanine (G), and uracil (U). Thus, terms “polynucleotide” and “nucleic acid” encompass single-stranded DNA: double-stranded DNA: multi-stranded DNA: single-stranded RNA: double-stranded RNA: multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Complementarity” refers to a first nucleic acid having a first sequence that allows it to “base pair,” “bind,” “anneal”, or “hybridize,” to a second nucleic acid. Binding may be affected by the amount of complementarity and certain external conditions such as ionic strength of the environment, temperature, etc. Base-pairing rules are well known in the art (A pairs with T in DNA, and with U in RNA; and G pairs with C). In some cases, RNA may include pairings where G may pair with U. Complementarity does not, in all cases, indicate complete or 100% complementarity. For example, complementarity may be less than 100% and more than about 60%. By “hybridizable” or “complementary” or “substantially complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T). A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.); G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package. Version 8 for Unix, Genetics Computer Group. University Research Park. Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones. The residues may also be modified prior to, or after incorporation into the polypeptide. In some embodiments, the polypeptides may be branched as well as linear.

“Binding” as used herein (e.g., with reference to an RNA-binding domain of a polypeptide, binding to a target nucleic acid, and the like) refers to a non-covalent interaction between macromolecules (e.g., between a protein and a nucleic acid; between a guide RNA and a target nucleic acid: and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶M, less than 10⁻⁷ M, less than 10⁻⁸ M, less than 10⁻⁹M, less than 10⁻¹⁰ M, less than 10⁻¹¹ M, less than 10⁻¹²M, less than 10⁻¹³ M, less than 10⁻¹⁴M, or less than 10⁻¹⁵M. “Affinity” refers to the strength of binding, increased binding affinity being correlated with a lower K_{d}.

“Cas protein” is a CRISPR associated protein. The presently disclosed Cas proteins possess a nuclease activity that may be activated upon binding of a target sequence to a guide RNA bound by the Cas protein. As disclosed in more detail below, the guide RNA may, with other sequences, comprise a crRNA, which may, in some embodiments, be processed from a pre-crRNA sequence. In an embodiment, the guide RNA sequence may include natural or synthetic nucleic acids, for example modified nucleic acids such as, without limitation, locked nucleic acids (LNA). 2′-o-methylated bases, or even ssDNA (single stranded DNA). Cas proteins may be from the Cas12 or Cas13 group, which may be derived from various sources known to those of skill in the art.

The Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof includes but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

“Coding sequences” are DNA sequences that encode polypeptide sequences or RNA sequences, for example guide RNAs. Coding sequences that encode polypeptides are first transcribed into RNA, which, in-turn, may encode the amino acid sequence of the polypeptide. Some RNA sequences, such as guide RNAs may not encode amino acid sequences.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

### Challenges in Monitoring Adherence to Antiretroviral Therapy

Over 40% of the estimated 1.4 million children living with HIV worldwide had access to antiretroviral therapy (ART) in 2023. Even among those with access, pediatric ART dosing is challenging to implement—often done by weight and without fixed dose combinations. Moreover, given the added safety constraints associated with implementing clinical trials among children, there are fewer approved ART regimens for children. And rates of non-adherence to medication among children are very high resulting in high rates of treatment failure and drug resistance. Dosing ARTs for children is challenging due to their rapid growth and developmental changes, which affect drug metabolism and pharmacokinetics. There is a growing need for age-appropriate formulations and precise dosing to ensure therapeutic efficacy and minimize adverse effects. The scarce amount of safety and pharmacokinetic data for children and neonates slows the approval of new treatments, which increases the risk of drug resistance.

Challenges with medication adherence compound the difficulty of pediatric dosing. Non-adherence arises due to factors such as complex medication regimens, side effects, inadequate support systems, and socioeconomic barriers. To ensure consistent and proper medication intake in children, many tailored strategies have been developed and piloted, such as caregiver support, simplified treatment regimens, and interventions addressing social and behavioral factors, but have been limited in the success of improving adherence. The efficacy of these interventions is often assessed with adherence monitoring strategies like surveys and pill counts, but these subjective measures do not always correlate with treatment outcomes. HIV viral load monitoring is a lagging indicator of non-adherence and may not indicate impending failure until symptoms and drug resistance have occurred.

HIV drug level feedback (DLF) provides objective information about medication adherence that correlates with treatment outcomes. Clinical trials, primarily among adults, have established drug levels that correspond to different adherence thresholds and treatment outcomes like virological failure. DLF has also been useful among other key populations such as adolescent girls and young women (AGYW), where both AGYW and their healthcare providers expresses a desire for DLF with concomitant increases in adherence and HIV prevention outcomes.

Despite the promise of HIV DLF, its use has largely been restricted to clinical and implementation trial settings because of the high cost and heavy instrumentation required to perform gold-standard liquid chromatography tandem mass spectrometry (LC-MS/MS) measurements. Although LC-MS/MS results were provided to AGYW and their healthcare providers in studies like MTN-034/REACH, the typical time to results was ˜56 days, significantly delaying interventions. Recently developed point-of-care (POC) tests, like the urine tenofovir (TFV) lateral flow assay (LFA) provide a rapid and inexpensive alternative and increased adherence to pre-exposure prophylaxis (PrEP) regimens among AGYW in Kenya. However, tenofovir-based regimens are not approved for use in children and LFAs have not yet been developed for the key antiretroviral drugs used in pediatric regimens, namely, azidothymidine (AZT), abacavir (ABC), and lamivudine (3TC).

There is a need to improve treatment outcomes for children living with HIV (CLWH). Adherence monitoring and drug level feedback has shown promise for improving ART treatment outcomes for CLWH, a population that remains challenging to treat due to difficulties in dosing and the limited number of approved ARTs. However, the gold-standard for drug level feedback remains centralized, has long turn-around times, and cannot be used in clinical settings.

Thus, there is a need for new systems and methods for monitoring HIV DLF among children in POC settings. Such systems and methods would find immediate use in monitoring and improving adherence to pediatric ART regimens and help in improving precision and personalization in pediatric ART dosing to increase efficacy and reduce toxicity.

### REverse Transcriptase ACTivity (REACT) Assays

Previously. REverse transcriptase ACTivity (REACT) assays were developed for rapid and minimally instrumented measurement of reverse transcriptase (RT) inhibitors—the backbone of HIV treatment and prevention regimens—as a function of the drugs' activity and demonstrated that REACT can measure clinically relevant concentrations of the active intracellular forms of RT inhibitors including TFV diphosphate (TFV-DP). 3TC triphosphate (3TC-TP), and AZT triphosphate (AZT-TP). It was also demonstrated that by designing custom DNA templates based on the chemical structure of nucleotide analog drugs and Watson-Crick-Franklin base pairing, one can measure the activity of co-administered drugs without cross-reactivity. Additionally, the capabilities of REACT assay were expanded to measure multiple non-nucleotide analog RT inhibitors with readout in a portable and inexpensive fluorescent reader.

However, despite the potential of REACT to provide a path to point of care (POC) HIV DLF, there are technical challenges that prevent its direct application to pediatric ART monitoring. Prior implementation of the assay relied on an intercalating dye to provide non-specific fluorescence when it binds to double-stranded DNA (dsDNA) synthesized by HIV RT. For drugs like TFV-DP that accumulate in RBCs. REACT can measure DNA synthesis with minimal interference from background genomic DNA (gDNA) since RBCs do not have a nucleus. However, all the approved antiretroviral drugs for children do not accumulate appreciably in RBCs and must be measured in peripheral blood mononuclear cells (PBMCs) where background gDNA would interfere with the assay signal. Furthermore, the intercalating dye non-specifically inhibited HIV RT activity and required endpoint addition, which introduces an additional timed assay step while preventing the acquisition of real-time assay information that could prove vital in assay design and optimization.

REACT with CRISPR Readout (REACTR) Assay

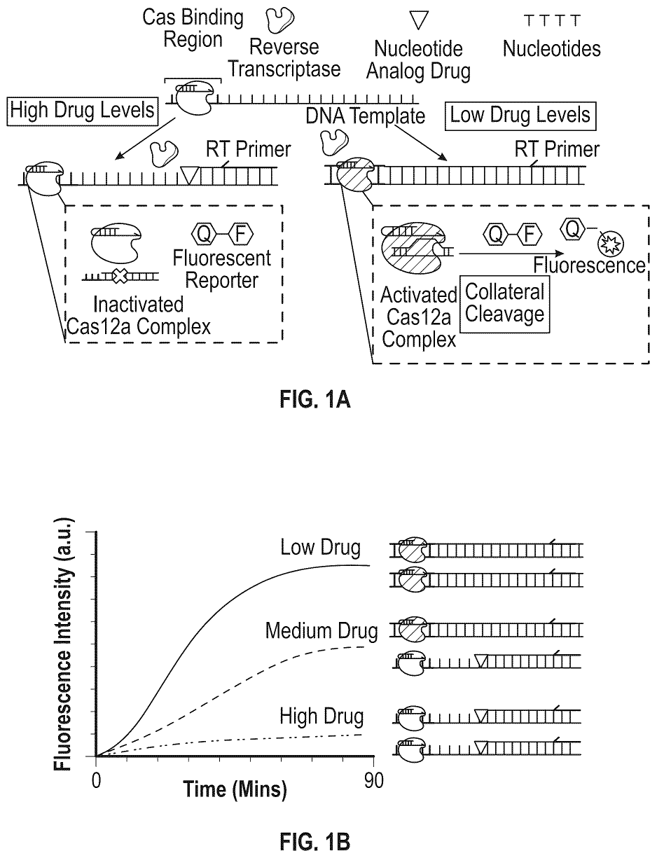

The present disclosure provides an assay that combines REACT with CRISPR readout (REACTR) assay where the non-specific intercalating dye readout is replaced by CRISPR Cas reporter complexes that target the sequence of synthesized DNA generated by HIV RT (FIGS. 1A-1B). REACTR leverages advances in CRISPR-based diagnostics that enable high sensitivity and specificity, versatility, and ease of use in measuring nucleic acid targets. Specifically. CRISPR-Cas12a is a powerful gene-editing tool that uses a Cas12a enzyme and a guide RNA (crRNA) to precisely target DNA at specific locations with attomolar accuracy. Additionally. Cas12a can cut nearby DNA or RNA after activation through its collateral cleavage activity, thus enabling fluorescent readout. Thus, the present disclosure demonstrates that REACTR can provide real-time information about HIV RT activity in the presence of AZT-TP and characterize the impact of design parameters like reaction time and DNA template length on REACTR's sensitivity. REACTR was also optimized to measure AZT-TP concentrations that are clinically relevant for ART adherence and dosing in children and validate assay performance in more complex sample matrices, including gDNA and PBMC lysate where our previous intercalating dye readout failed.

The REACTR assay has the ability to detect drug levels in 30 minutes to improve long-term adherence. The present disclosure demonstrates REACTR's ability to quantify clinically relevant concentrations of AZT-TP, a main drug in pediatric regimens, and the influence of DNA template length on REACTR's sensitivity. REACTR's performance has also been validated in the presence of genomic DNA and peripheral mononuclear blood cell lysate, as AZT-TP accumulates in white blood cells, showing no impact on performance. REACTR's ability to monitor drug levels could improve treatment outcomes of CLWH by preventing drug resistance and improving therapeutic dosing.

Accordingly, the present disclosure provides a novel platform that can detect AZT-TP levels in the presence of genomic DNA and PBMC lysate. REACTR was further characterized to find the optimal template length and incubation period. REACTR showed the same sensitivity to AZT-TP in genomic DNA and PBMC lysate indicating the presence did not affect the REACTR assay's integrity. The presently disclosed novel approach allowed for nucleotide analog detection accumulates in white blood cells. The present disclosure demonstrates the feasibility of using CRISPR-based assays for DLF and adherence assays. By using the CRISPR Cas12a system, a whole new class of HIV medications could be detected in 30 minutes that can be used for monitoring and long-term adherence testing for children living with HIV.

Further optimization of REACTR includes but is not limited to more point-of-care friendly formats, such as using a portable fluorescent reader, optimizing REACTR in whole blood, and lyophilizing the reagents. Additionally, the presently disclosed methods may be utilized to measure other types of reverse transcriptase inhibitors, including other nucleotide analogs and non-nucleoside reverse transcriptase inhibitors. Currently. REACTR is optimized for T-analog NRTIs, like AZT, and can be expanded to other nucleotide analogs, like tenofovir or emtricitabine. The disclosed DNA template can be redesigned so it is specific to NRTIs allowing REACTR to be tailored to specific ART regimens.

REACTR holds promise for enhancing therapeutic outcomes among children living with HIV. The ability to monitor drug levels quickly and accurately ensures that pediatric patients maintain therapeutic drug concentrations, which is useful for preventing the development of resistance and managing side effects. REACTR assay could significantly improve access to monitoring in low-resource settings, where many affected children reside. Overall. REACTR represents a significant step forward in improving the management of HIV for children, by providing a rapid and adaptable method for monitoring drug adherence.

### CRISPR Effector System

The CRISPR effector systems disclosed herein comprise (i) at least one CRISPR effector protein having collateral cleavage activity; and (ii) at least one sgRNA that directs the at least one CRISPR effector protein to specifically bind to a target nucleic acid sequence.

### CRISPR Cas or CRISPR Effector Proteins

CRISPR/Cas system (Clustered regularly interspaced short palindromic repeats/CRISPR-associated protein) is an adaptive immune defense mechanism against foreign gene invasion in prokaryotes. Evolved by bacteria and archaea in the process of defending against the invasion of foreign viruses and phages. The system can integrate DNA fragments invaded by foreign aid into the CRISPR site and then guide the Cas endonuclease to cut the foreign DNA sequence through the corresponding CRISPR RNAs (crRNAs), thereby resisting the invasion of viruses or phages. CRISPR/Cas gene clusters consist of a series of genes encoding Cas proteins (Cas1, Cas2, Cas4 and effector proteins such as Cas9. Cpf1, etc.) and CRISPR sequences. The CRISPR sequence consists of a leader sequence (leader), many short and conserved repeat regions (repeat) and spacer (spacer). Repeated sequence regions contain palindromic sequences that can form hairpin structures. The spacer is the foreign DNA sequence captured by the host. These captured foreign DNA sequences are equivalent to the “foreign objects” of the immune system. When these foreign genetic materials invade the host again, bacteria begin to transcribe CRISPR to form the primary transcription product pre-crRNA, which is then processed by ribonuclease or Cas protein. The mature crRNA is cleaved within the repeat sequence site and forms a ribonucleoprotein complex with a specific CRISPR effector protein, which recognizes and cleaves the exogenous DNA that can complement the crRNA, causing double-strand breaks and triggering the self-repair of the host cell.

In general, a CRISPR system or CRISPR effector proteins as used herein refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system)

The non-specific “collateral” cleavage activity of some CRISPR-associated proteins may be dormant until being activated by the binding of other factors to the protein or protein complex. As such, Cas13 or Cas12 enzymes can be programmed with a guide RNA that recognizes a desired target sequence, activating a non-specific RNase or DNase activity. This can be used to release a detectable label, such as a quenched fluorescent reporter, leading to a detectable signal such as fluorescence. See, e.g., Li et al. (2019) Trends in Biotech. 37 (7): 730-743; Petri & Pattanayak (2018) The CRISPR Journal 1 (3): 209-211; Gootenberg et al. (2017) Science 356 (6336): 438-442; and U.S. Publication Nos. 20180274017 and 20190241954.

According to the composition of the Cas gene and the number of effector proteins. CRISPR is divided into 2 types and 5 types, with a total of 16 subtypes. Type 1 is the CRISPR/Cas system that uses multiple effector protein complexes to interfere with target genes, including types I, III and IV; type 2 is the CRISPR/Cas system that uses a single effector protein to interfere with target genes, including type II and type V. At present, the most widely studied and used type II is the CRISPR/Cas9 system.

Type V CRISPR/Cas proteins, e.g., Cas12 proteins such as Cpf1 (Cas12a) and C2c1 (Cas12b) can promiscuously cleave non-targeted single stranded DNA (ssDNA) once activated by detection of a target DNA (double or single stranded). Once a type V CRISPR/Cas effector protein (e.g., a Cas12 protein such as Cas12a, Cas12b, Cas12c, Cas12d, Cas12e) is activated by a guide RNA, which occurs when the guide RNA hybridizes to a target sequence of a target DNA (i.e., the sample includes the targeted DNA), the protein becomes a nuclease that promiscuously cleaves ssDNAs (i.e., the nuclease cleaves non-target ssDNAs, i.e., ssDNAs to which the guide sequence of the guide RNA does not hybridize). Thus, when the target DNA is present in the sample (e.g., in some cases above a threshold amount), the result is cleavage of ssDNAs in the sample, which can be detected using any convenient detection method (e.g., using a labeled single stranded detector DNA). The term “Cas12a” (also referred to as “Cpf1”): is an endonuclease in the CRISPR system, Cas12a is characterized by both cis and trans single-stranded DNA cleavage activities. Cas12a is very suitable for rapid nucleic acid detection research.

In some embodiments. “CRISPR proteins” or “CRISPR effector protein” or “CRISPR enzymes” refers to Class V CRISPR effector proteins including but not limited to a Cas12 protein such as Cas12a (formerly known as Cpf1), Cas12b, Cas12c, Cas12d, Cas12e. In one embodiment, the CRISPR effector proteins described herein are preferably Cpf1 effector proteins.

The methods disclosed herein refer to a technology that uses a CRISPR/Cas system or CRISPR/Cas detection complex, for example, CRISPR-Cas12a to specifically recognize and cut the target gene, and non-specifically cut a ssDNA fluorescent reporter molecule, and detect the target nucleic acid molecule with the help of a detectable signal, for example, a fluorescent signal or a colorimetric signal.

The term “Cas12a” (also referred to as “Cpf1”) is an endonuclease in the CRISPR system. Cas12a is characterized by both cis and trans single-stranded DNA cleavage activities, Cas12a is very suitable for rapid nucleic acid detection.

The Type V CRISPR/Cas effector protein (e.g., a Cas12 protein such as Cas12a, Cas12b. Cas12c, Cas12d, Cas12e) can be provided as a protein or as a nucleic acid encoding the protein (e.g., an mRNA, a DNA such as a recombinant expression vector). In some cases, two or more (e.g., 3 or more, 4 or more, 5 or more, or 6 or more) guide RNAs can be provided by (e.g., using a precursor guide RNA array, which can be cleaved by the Type V CRISPR/Cas effector protein into individual (“mature”) guide RNAs).

### Guide RNA

As used herein. “guide RNA” or “gRNA” refers to the non-coding RNA sequence that binds to the complementary target DNA sequence to guide the CRISPR-Cas system in close contact with the target DNA strand. As used herein, the term “sgRNA”: refers to a small guide molecule capable of targeting specific nucleic acid molecules and activating Cas12a collateral cleavage activity. The guide RNA can be provided as RNA or as a nucleic acid encoding the guide RNA (e.g., a DNA such as a recombinant expression vector).

### Reporter Molecules and Nucleic Acid Detection Systems

The nucleic acid detection systems disclosed herein comprise of a reporter molecule or a nucleic acid probe. In certain embodiments, the term “reporter molecule” or “nucleic acid probe” refers to a certain length of single-stranded DNA (ssDNA) comprising a target site that is preferentially cleaved by collateral cleavage activity of a CRISPR effector protein. In some embodiments, the ssDNA further comprises a fluorescent group and a quencher group added to the bases at the 5′ and 3′ ends, respectively, to obtain the ssDNA-reporter/nucleic acid probe. Its function in the CRISPR effector-based detection system is that when ssDNA is non-specifically cleaved by CRISPR effector protein, for example, Cas12a, the fluorophore and the quencher group are separated, and the quencher group releases the blocking effect on the fluorophore. Fluorescence intensity changes can be detected by means of fluorescence signal detection equipment. In some embodiments, the present disclosure provides a nucleic acid probe comprising a detectable label and detection systems, wherein the nucleic acid probe comprises a site preferentially cleaved by collateral cleavage activity of the CRISPR effector protein.

As used herein a “CRISPR/Cas detection complex” refers to a complex comprising a guide RNA and a Cas effector enzyme configured to cleave a labeled nucleic acid reporter in response to a drug-responsive nucleic acid signal.

In some embodiments, the methods, devices, or systems disclosed herein include a step of detecting or a system for detecting (e.g., measuring a detectable signal produced by the collateral cleavage of the nucleic acid probe. In an exemplary embodiment, the collateral cleavage of the nucleic acid probe is mediated by a CRISPR effector protein. In some embodiments, the CRISPR effector protein is a Type V CRISPR/Cas effector protein (e.g., a Cas12 protein such as Cas12a, Cas12b, Cas12c, Cas12d, Cas12e)-mediated ssDNA cleavage). Because a Type V CRISPR/Cas effector protein (e.g., a Cas12 protein such as Cas12a, Cas12b, Cas12c, Cas12d, Cas12e) cleaves non-targeted ssDNA once activated, which occurs when a guide RNA hybridizes with a target DNA in the presence of a Type V CRISPR/Cas effector protein (e.g., a Cas12 protein such as Cas12a, Cas12b, Cas12c, Cas12d, Cas12e), a detectable signal can be any signal that is produced when ssDNA is cleaved.

Detectable labels include but are not limited to fluorescent molecules and the like. For example, in some cases the step of measuring can include one or more of: gold nanoparticle based detection (e.g., see Xu et al., Angew Chem Int Ed Engl. 2007: 46 (19): 3468-70; and Xia et al., Proc Natl Acad Sci USA. 2010 Jun. 15; 107 (24): 10837-41), fluorescence polarization, colloid phase transition/dispersion (e.g., Baksh et al., Nature, 2004 Jan. 8: 427 (6970): 139-41), electrochemical detection, semiconductor-based sensing (e.g., Rothberg et al., Nature, 2011 Jul. 20: 475 (7356): 348-52; e.g., one could use a phosphatase to generate a pH change after ssDNA cleavage reactions, by opening 2′-3′ cyclic phosphates, and by releasing inorganic phosphate into solution), and detection of a labeled detector ssDNA (see elsewhere herein for more details).

The readout of such detection methods can be any convenient readout. Examples of possible readouts include but are not limited to: a measured amount of detectable fluorescent signal: a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

The term “visual detection” means that if a fluorescent signal is observed, it needs to be detected with the help of a fluorescence detection device; and if the color is observed to change, it can be directly visualized with the naked eye without the need for an instrument.

Accordingly, in one aspect, the present disclosure provides a system for detecting a target enzyme, the system comprising: (a) a CRISPR effector system; and (b) a detection system. In certain aspects, the CRISPR effector system comprises (i) at least one CRISPR effector protein having collateral cleavage activity; and (ii) at least one sgRNA that directs the at least one CRISPR effector protein to specifically bind to a target nucleic acid sequence. In certain aspects, the detection system comprises a nucleic acid probe comprising a detectable label; and a site preferentially cleavable by collateral cleavage activity of the CRISPR effector protein.

In some embodiments, the target nucleic acid sequence is a sequence generated/synthesized by the activity of the target enzyme. In some embodiments, the nucleic acid probe comprises a site cleavable by collateral cleavage activity of the at least one CRISPR effector protein. In an embodiment, when the nucleic acid probe is cleaved a detectable signal is generated from the detectable label. In some embodiments, when the nucleic acid probe is cleaved a detectable signal is removed from the detectable label. In some embodiments, the system further comprises reagents for generation and amplification of the target nucleic acid, wherein the reagents comprise: (i) a nucleic acid template; (ii) dNTPs; and (iii) primers.

In some embodiments, the system employs Cas12a (Cpf1), guided by a crRNA complementary to a DNA-based target signal generated by the activity of the target enzyme to be detected. In other embodiments, Cas13a (C2c2) or Cas13b is utilized for detection of RNA-based transduced signals. For highly compact formats or low-abundance targets, Cas14a or CasDx1 may be used for increased sensitivity due to reduced non-specific activity.

In some embodiments, the at least one CRISPR effector protein comprises a Cas12a complex. In an embodiment, the at least one CRISPR effector protein comprises a Cas protein with similar bypass/collateral cleavage activity as Cas12a. In some embodiments, the sgRNA is a crRNA molecule.

In an embodiment, when the target enzyme to be detected is not active or is absent, the target nucleic acid sequence is not generated, the CRISPR effector system is inactive, and the nucleic acid probe is not cleaved by the collateral cleavage activity of the at least one CRISPR effector protein to generate the detectable signal from the detectable label or to remove the detectable signal from the detectable label. In an embodiment, when the target enzyme is active or present, the target nucleic acid sequence is generated, the CRISPR effector system is active, and the nucleic acid probe is cleaved by the collateral cleavage activity of the at least one CRISPR effector protein to generate the detectable signal or to remove the detectable signal from the detectable label. In some embodiments, the generation or removal of the detectable signal is detected using a detection equipment/instrument. In an embodiment, the generation or removal of the detectable signal is visually detected by naked eyes without an instrument/detection equipment.

In some embodiments, the nucleic acid probe comprises a first molecule attached to the 5′ end and a second molecule attached to the 3′ ends of an oligo or vice-versa, or wherein the nucleic acid probe comprises a first molecule attached on any base of the oligo and a second molecule attached to a different base of the oligo. In some embodiments, the nucleic acid probe is an RNA or DNA oligonucleotide. In an embodiment, the nucleic acid probe is a single-stranded RNA or a single-stranded DNA oligonucleotide, and wherein the first molecule and the second molecule are covalently attached on the base of the single-stranded RNA or single-stranded DNA oligonucleotide. In some embodiments, the first molecule is a fluorescent molecule selected from FITC, Cy5, FAM, Texas Red, and Cy7. In some embodiments, the second molecule is a fluorescent quencher, wherein the fluorescent quencher is selected from BHQ-1, BHQ-2, Dabcyl, and Iowa Black FQ.

In some embodiments, the disclosed system utilizes a CRISPR/Cas effector enzyme capable of sequence-specific cleavage of a nucleic acid reporter/probe in response to generation by a target enzyme of a target nucleic acid that correlates with the presence or concentration or activity of a target enzyme. In some embodiments, the target enzyme activity or concentration correlates with the concentration of a drug or its metabolite. The system may employ Cas12, Cas13, or other nucleic acid-guided enzymes exhibiting collateral cleavage activity. In exemplary embodiments, a nucleic acid probe/reporter is used to transduce the presence of or the activity of a target enzyme into a detectable nucleic acid sequence, which subsequently activates the collateral cleavage activity of the CRISPR complex.

In another aspect, the present disclosure provides a visual detection system for detecting/monitoring enzymatic activity of a target enzyme. In some embodiments, the system comprises (a) a CRISPR effector system; and (b) a detection system comprising a nucleic acid probe comprising a visually detectable label; and a site preferentially cleaved by collateral cleavage activity of the CRISPR effector protein. In some embodiments, the CRISPR effector system comprises: (i) at least one CRISPR effector protein, said CRISPR effector protein being Cas12a or a Cas protein with similar collateral cleavage activity as Cas12a; and (ii) at least one sgRNA that directs the at least one CRISPR effector protein to specifically bind to a target nucleic acid sequence. In some embodiments, the target nucleic acid sequence is a sequence generated/synthesized by the enzymatic activity of the target enzyme. In an embodiment, a visually detectable signal is generated when the nucleic acid probe is cleaved by the collateral cleavage activity of the at least one CRISPR effector protein. In some embodiments, a visually detectable signal is removed when the nucleic acid probe is cleaved by the collateral cleavage activity of the at least one CRISPR effector protein.

In an embodiment, the systems disclosed herein are present in a lateral flow format.

The present disclosure also relates to systems and methods for monitoring drug levels in a biological sample using a lateral flow assay (LFA) incorporating the CRISPR/Cas-effector system and the detector system disclosed herein. In certain embodiments, the disclosed systems are configured to provide real-time or near real-time feedback on drug concentrations in a subject, which can be further integrated with digital monitoring platforms to support adherence assessment and therapeutic optimization.

In certain aspects, the present disclosure provides a lateral flow assay (LFA) device/system for detecting/monitoring enzymatic activity of a target enzyme in a biological sample. In some embodiments, the enzymatic activity of the target enzyme correlates with presence, absence, or concentration of a drug and/or its metabolite in the biological sample. In some embodiments, the device/system comprises a substrate comprising a first end. In an embodiment, the first end comprises: a sample loading portion; and a first region.

In some embodiments, the first region comprises: a CRISPR effector system: a detection system; and a first capture region comprising a first binding agent. In some embodiments, the CRISPR effector system comprises at least one CRSPR effector protein having collateral cleavage activity; and at least one sgRNA that directs the at least one CRISPR effector protein to specifically bind to a target nucleic acid sequence. In some embodiments, the detection system comprises a nucleic acid probe comprising a detectable label; and a site preferentially cleavable by collateral cleavage activity of the CRISPR effector protein.

In some embodiments, the target nucleic acid sequence is a sequence generated/synthesized by the enzymatic activity of the target enzyme. In an embodiment, the nucleic acid probe is cleavable by collateral cleavage activity of the CRISPR effector system. In some embodiments, when the nucleic acid probe is cleaved a detectable signal is generated from the detectable label. In some embodiments, when the nucleic acid probe is cleaved a detectable signal is removed from the detectable label. In some embodiments, the sample loading portion further comprises reagents for generation of the target nucleic acid, wherein the reagents comprise: (i) a nucleic acid template; (ii) dNTPs; and (iii) primers.

In some embodiments, the first capture region is proximate to and on the same end of the lateral flow substrate as the sample loading portion. In an embodiment, the first capture region comprises a first binding agent that specifically binds the first molecule of the nucleic acid probe. In some embodiments, the device/system further comprises a second capture region. In an embodiment, the second capture region is located towards the opposite end of the lateral flow substrate from the first binding region. In an embodiment, the second capture region optionally comprises a second binding agent that specifically binds the second molecule of the nucleic acid probe.

In some embodiments, the sample is loaded in the sample loading portion of the substrate. In an embodiment, the sample flows from the sample loading portion of the substrate to the first and optionally the second capture regions. In some embodiments, when the target enzyme is not active or is absent in the sample, the target nucleic acid sequence is not generated, the CRISPR effector system is inactive, and the nucleic acid probe is not cleaved by the collateral cleavage activity of the at least one CRISPR effector protein to generate a visually detectable signal from the detectable label or to remove a visually detectable signal from the detectable label. In some embodiments, when the target enzyme is active or present, the target nucleic acid sequence is generated, the CRISPR effector system is active, and the nucleic acid probe is cleaved by collateral cleavage activity of the CRISPR effector protein to generate a visually detectable signal from the detectable label or to remove a visually detectable signal from the detectable label.

In some embodiments, the sample is a biological sample; wherein the biological sample is optionally a blood, plasma, serum, urine, saliva, mucous, cervicovaginal fluid, lymph fluid, synovial fluid, ascites, pleural effusion, seroma, cerebrospinal fluid, sputum, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

In some embodiments, the target enzyme is a pathogen-derived enzyme. In some embodiments, the systems and methods disclosed herein detect the pathogen-derived enzyme in response to an anti-pathogenic agent. In certain embodiments, the pathogen-derived enzyme is a reverse transcriptase enzyme or a retroviral integrase of the Human immunodeficiency virus (HIV), and the anti-pathogenic agent is an Antiretroviral therapeutic (ART) agent. The anti-pathogenic agent can be added to the biological sample as a reagent or be present in the biological sample. Signal generated or removed in the presence of anti-pathogenic agent can indicate resistance of the target enzyme against the anti-pathogenic agent. The difference in signal generated or removed in the presence of anti-pathogenic agent versus in the absence of anti-pathogenic agent can indicate resistance of the target enzyme to the anti-pathogenic agent.

The Lateral Flow Assay (LFA) architecture can include a sample pad, conjugate pad, nitrocellulose membrane, and absorbent pad arranged in fluid communication. In use, a sample such as blood, saliva, or urine is applied to the sample pad. The sample migrates by capillary action through the conjugate pad, where a dried CRISPR/Cas detection complex or the CRISPR effector proteins is rehydrated. Upon encountering the target enzyme/drug-responsive nucleic acid signal, the CRISPR/Cas enzyme becomes activated and cleaves a nucleic acid probe or a reporter molecule comprising a fluorophore-quencher or a biotin-fluorescein, or another dual label construct.

The cleaved reporter is then captured and visualized along a test and control line on the nitrocellulose membrane. In colorimetric embodiments, the cleavage event results in release or accumulation of a signal moiety (e.g., gold nanoparticles, latex beads, or enzymes), producing a visible line whose intensity correlates with drug concentration. In other embodiments, the output signal is fluorescent, chemiluminescent, or electrochemical, enabling enhanced sensitivity or quantitative measurement.

The CRISPR/Cas complex may be pre-assembled and lyophilized onto the conjugate pad or stored in a separate chamber for activation upon sample addition. The nucleic acid reporter may be designed for single-use cleavage, ensuring minimal background signal. In certain embodiments, the system includes an internal control line to validate proper sample migration and reagent rehydration.

In some embodiments, the system employs Cas12a (Cpf1), guided by a crRNA complementary to a DNA-based target signal generated through the activity of a target enzyme. In other embodiments, Cas13a (C2c2) or Cas13b is utilized for detection of RNA-based transduced signals. For highly compact formats or low-abundance targets, Cas14a (also known as Cas12f) or CasDx1 may be used for increased sensitivity due to reduced non-specific activity.

The LFA system may be adapted for various therapeutic agents, including but not limited to antiretroviral drugs, antibiotics, immunosuppressants, or chemotherapeutics. In certain embodiments, a panel of drug-specific nucleic acid probes-CRISPR sensors can be multiplexed on a single lateral flow strip to enable simultaneous measurement of multiple drug levels or metabolites.

In particular embodiments, the target enzyme-responsive nucleic acid probe detects a small-molecule therapeutic such as an antiretroviral, antibiotic, or chemotherapeutic. Examples include, without limitation; tenofovir disoproxil fumarate, efavirenz, dolutegravir, lamivudine, and emtricitabine; rifampicin; 5-fluorouracil, cytarabine.

In certain embodiments, the nucleic acid probe binds the drug molecule or the metabolite of the drug molecule directly, while in others, an enzymatic or competitive displacement mechanism is used to link the drug presence to nucleic acid generation for CRISPR activation.

In further embodiments, multiple nucleic acid probe-CRISPR effector modules are immobilized on distinct regions of a single LFA strip, enabling multiplexed detection of several drugs or drug classes from one sample. Each module may contain a CRISPR effector tuned to a specific trigger sequence, with distinct reporter fluorophores or colorimetric tags to differentiate analytes.

In some embodiments, the disclosed system includes a digital reader or optical detection module configured to capture, process, and transmit the assay signal. The reader may be integrated within a smartphone attachment, a standalone portable device, or an embedded optical sensor. Image analysis software may quantify the intensity or spectral features of the test and control lines to derive a drug concentration value. The data may then be transmitted via a secure communication channel to a cloud-based or locally stored digital adherence platform.

The digital integration component can include algorithms for temporal tracking of drug exposure, threshold-based adherence alerts, and visualization dashboards accessible to healthcare providers or caregivers. In some embodiments, the reader can be configured to automatically calibrate assay results based on environmental parameters (e.g., temperature, humidity) or prior device calibration data to ensure analytical consistency.

In some embodiments, the LFA and digital module are housed in a single disposable or semi-reusable device or cartridge. The cartridge may include sample metering microchannels, air vents, and desiccant compartments to ensure reliable flow and storage stability. The digital reader can include a calibration module that automatically adjusts for environmental variables such as illumination, temperature, or humidity to maintain assay accuracy.

In some embodiments, the assay operates semi-quantitatively, providing threshold-based readouts (e.g., above or below therapeutic range). In other embodiments, quantitative determination is achieved through ratiometric analysis of test and control signal intensities, or through calibration curves stored in the associated digital system.

In further embodiments, the assay and reader are integrated into a closed cartridge system to minimize biohazard exposure and user variability. The cartridge may include microfluidic features to meter sample volumes and ensure uniform flow. Power for the detection or communication module may be provided via battery, near-field communication (NFC), or wireless charging.

In certain embodiments, the system disclosed herein is coupled with a dosing feedback loop. The digital platform can analyze temporal trends in drug levels and generate adherence feedback, reminders, or dosage adjustment recommendations according to pre-set clinical algorithms. In this manner, the disclosed technology provides a portable, low-cost, and digitally connected molecular diagnostic platform that bridges pharmacologic monitoring with real-time behavioral feedback.

In some embodiments, the present disclosure provides a system for monitoring a drug level in a biological sample, comprising (a) a lateral flow assay (LFA) device comprising a sample pad, a conjugate pad, a nitrocellulose membrane, and an absorbent pad arranged in fluid communication; (b) a CRISPR/Cas detection complex disposed on or within the conjugate pad, wherein the CRISPR/Cas detection complex comprises a guide RNA and a Cas effector enzyme configured to cleave a labeled nucleic acid reporter in response to activation by a drug-responsive nucleic acid signal: (c) a reporter configured to produce a detectable signal upon cleavage; and (d) a digital reader configured to detect the signal and generate an output indicative of the concentration of the drug in the biological sample. In certain embodiments, the drug-responsive nucleic acid signal comprises absence or low concentrations of the drug level in the biological sample. In some embodiments, the detectable signal is colorimetric, fluorescent, chemiluminescent, or electrochemical. In certain embodiments, the digital reader comprises an optical sensor configured to detect reflected light, fluorescence intensity, or luminescence from a test line and a control line on the lateral flow strip. In some embodiments, the digital reader includes a processor configured to normalize and analyze the signal intensity using a calibration curve or algorithm to compute a quantitative drug concentration.

In some embodiments, the present disclosure provides system for integrated drug adherence monitoring comprising (a) a CRISPR-based lateral flow assay configured to detect a drug or metabolite in a biological sample; (b) a digital reader configured to quantify the assay signal; and (c) a software platform configured to process and store temporal drug level data, compare measured concentrations to expected pharmacokinetic profiles, and provide adherence feedback or dosing recommendations.

In yet another aspect, the present disclosure provides a point of care method for monitoring adherence to a therapy targeting a disease-associated enzyme in a subject. In some embodiments, the method comprises: (i) obtaining a biological sample from the subject; (ii) contacting the biological sample with (a) a CRISPR effector system; and (b) a nucleic acid probe comprising a detectable label. In some embodiments, the CRISPR effector system comprises: (1) at least one CRISPR effector protein having collateral cleavage activity; and (2) at least one sgRNA that directs the at least one CRISPR effector protein to specifically bind to a target nucleic acid sequence. In some embodiments, the target nucleic acid sequence is a sequence generated/synthesized by the activity of the disease-associated enzyme. In an embodiment, the nucleic acid probe is cleavable by collateral cleavage activity of the at least one CRISPR effector protein. In some embodiments, when the nucleic acid probe is cleaved a detectable signal is generated from the detectable label. In some embodiments, when the nucleic acid probe is cleaved a detectable signal is removed from the detectable label. In some embodiments, the method further comprises measuring the detectable signal generated or removed from the detectable label; and determining a level of adherence to the therapy based on the measured detectable signal. In some embodiments, the method further comprises adding reagents for generation of the target nucleic acid, wherein the reagents comprise: (i) a nucleic acid template; (ii) dNTPs; and (iii) primers.

In some embodiments, the disease-associated enzyme is reverse transcriptase of Human immunodeficiency virus (HIV). In an embodiment, the disease-associated enzyme is an integrase of Human immunodeficiency virus (HIV). In some embodiments, the therapy comprises an antiretroviral therapy (ART). In an embodiment, the ART comprises tenofovir-diphosphate (TFV-DP).

In yet another embodiment, the present disclosure provides a method for determining a drug concentration in a biological sample, comprising (a) introducing the biological sample to a lateral flow assay comprising a CRISPR/Cas detection complex and a labeled reporter; (b) activating the CRISPR/Cas detection complex via a drug-responsive nucleic acid signal that correlates with the presence or concentration of the drug; (c) detecting a cleavage event of the reporter to generate a measurable signal; and (d) processing the measurable signal using a digital reader to determine or estimate the drug concentration and optionally transmitting the result to a remote or local data management platform.

The disclosed systems and methods thus provide a portable, low-cost, and digitally connected platform for monitoring drug levels in a manner suitable for use in clinical settings, remote monitoring, or resource-limited environments. By integrating CRISPR-based molecular specificity with the simplicity of a lateral flow format and digital quantification, the platform facilitates individualized adherence monitoring and data-driven therapeutic management.

### EMBODIMENTS

Also provided herein are the following non-limiting embodiments.
1. A system for detecting a target enzyme, the system comprising: (a) a CRISPR effector system comprising: (i) at least one CRISPR effector protein having collateral cleavage activity; and (ii) at least one sgRNA that directs the at least one CRISPR effector protein to specifically bind to a target nucleic acid sequence; and (b) a detection system comprising a nucleic acid probe comprising a detectable label, wherein the target nucleic acid sequence is a sequence generated/synthesized by the activity of the target enzyme, and wherein the nucleic acid probe is cleavable by collateral cleavage activity of the at least one CRISPR effector protein, and wherein when the nucleic acid probe is cleaved a detectable signal is generated from the detectable label or wherein when the nucleic acid probe is cleaved a detectable signal is removed from the detectable label.
2. The system of embodiment 1, wherein the system further comprises reagents for generation of the target nucleic acid, and wherein the reagents comprise: (i) a nucleic acid template; (ii) dNTPs; and (iii) primers.
3. The system of embodiment 1 or embodiment 2, wherein the at least one CRISPR effector protein comprises one or more thermostable Cas proteins possessing collateral activity, and wherein the one or more Cas protein is a Type V Cas.
4. The system of embodiment 1 or embodiment 2, wherein the at least one CRISPR effector protein comprises a Cas 12a protein or a protein with similar bypass/collateral cleavage activity as Cas12a.
5. The system of embodiment 3 or embodiment 4, wherein the at least one CRISPR effector protein sgRNA is a crRNA molecule.
6. The system of any one of embodiments 1-5, wherein when the target enzyme is not active or is absent, the target nucleic acid sequence is not generated, the CRISPR effector system is inactive, and the nucleic acid probe is not cleaved by the collateral cleavage activity of the at least one CRISPR effector protein to generate the detectable signal from the detectable label or to remove the detectable signal from the detectable label.
7. The system of any one of embodiments 1-5, wherein when the target enzyme is active or present, the target nucleic acid sequence is generated, the CRISPR effector system is active, and the nucleic acid probe is cleaved by the collateral cleavage activity of the at least one CRISPR effector protein to generate the detectable signal or to remove the detectable signal from the detectable label.
8. The system of any one of embodiments 1-7, wherein the generation or removal of the detectable signal is detected using a detection equipment/instrument.
9. The system of any one of embodiments 1-7, wherein the generation or removal of the detectable signal is visually detected by naked eyes without an instrument/detection equipment.
10. The system of any one of embodiments 1-9, wherein the nucleic acid probe comprises a first molecule attached to the 5′ end and a second molecule attached to the 3′ ends of an oligo or vice-versa, or wherein the nucleic acid probe comprises a first molecule attached on any base of the oligo and a second molecule attached to a different base of the oligo.
11. The system of embodiment 10, wherein the nucleic acid probe is an RNA or DNA oligonucleotide.
12. The system of embodiment 11, wherein the nucleic acid probe is a single-stranded RNA or a single-stranded DNA oligonucleotide, and wherein the first molecule and the second molecule are covalently attached on the base of the single-stranded RNA or single-stranded DNA oligonucleotide.
13. The system of any one of embodiments 10-12, wherein the first molecule is a fluorescent molecule selected from FITC, Cy5, FAM, Texas Red, and Cy7.
14. The system of any one of embodiments 10-13, wherein the second molecule is a fluorescent quencher, wherein the fluorescent quencher is selected from FAM. Biotin, digoxin, and dUTP.
15. The system of any one of embodiments 1-14, wherein the system is present in a lateral flow format.
16. The system of any one of embodiments 1-15, wherein the target enzyme is a pathogen-derived enzyme, and wherein the system detects the activity of the pathogen-derived enzyme following exposure to an anti-pathogenic agent.
17. The system of embodiment 16, wherein the pathogen-derived enzyme is a reverse transcriptase enzyme or a retroviral integrase of the Human immunodeficiency virus (HIV), and wherein the anti-pathogenic agent is an Antiretroviral therapeutic (ART) agent.
18. A visual detection system for detecting/monitoring enzymatic activity of a target enzyme, the system comprising: (a) a CRISPR effector system comprising: (i) at least one CRISPR effector protein, said CRISPR effector protein being Cas12a or a Cas protein with similar collateral cleavage activity as Cas12a; and (ii) at least one sgRNA that directs the at least one CRISPR effector protein to specifically bind to a target nucleic acid sequence; and (b) a nucleic acid probe comprising a visually detectable label, wherein the target nucleic acid sequence is a sequence generated/synthesized by the enzymatic activity of the target enzyme, and wherein a visually detectable signal is generated when the nucleic acid probe is cleaved by the collateral cleavage activity of the at least one CRISPR effector protein, or wherein a visually detectable signal is removed when the nucleic acid probe is cleaved by the collateral cleavage activity of the at least one CRISPR effector protein.
19. The visual detection system of embodiment 18, wherein the system further comprises reagents for generation of the target nucleic acid, wherein the reagents comprise: (i) a nucleic acid template; (ii) dNTPs; and (iii) primers.
20. The visual detection system of embodiment 18 or embodiment 19, wherein the sgRNA is a crRNA molecule.
21. The visual detection system of any one of embodiments 18-20, wherein when the target enzyme is not active, the target nucleic acid sequence is not generated, the CRISPR effector system is inactive, and the nucleic acid probe is not cleaved by the collateral cleavage activity of the at least one CRISPR effector protein to generate the visually detectable signal or to remove the visually detectable signal.
22. The visual detection system of any one of embodiments 18-20, wherein when the target enzyme is active, the target nucleic acid sequence is generated, the CRISPR effector system is active, and the nucleic acid probe is cleaved by the collateral cleavage activity of the at least one CRISPR effector protein to generate a visually detectable signal or to remove the visually detectable signal.
23. The visual detection system of any one of embodiments 18-22, wherein the visually detectable signal is a fluorescence signal detectable by naked eye or by means of fluorescence detection equipment or wherein the visually detectable signal is a color change detectable directly by naked eye.
24. The visual detection system of any one of embodiments 18-22, wherein the nucleic acid probe comprises a first molecule attached to the 5′ end and a second molecule attached to the 3′ ends of an oligo or vice-versa, or wherein the nucleic acid probe comprises a first molecule attached on any base an oligo and a second molecule attached to a different base of the oligo.
25. The visual detection system of embodiment 24, wherein the nucleic acid probe is an RNA or DNA oligonucleotide.
26. The visual detection system of embodiment 24 or embodiment 25, wherein the nucleic acid probe is a single-stranded RNA or a single-stranded DNA oligo, and the first molecule and the second molecule are covalently attached on a base of the oligo.
27. The visual detection system of any one of embodiments 24-26, wherein the first molecule is a fluorescent molecule selected from FITC, Cy5, FAM, Texas Red, and Cy7.
28. The visual detection system of any one of embodiments 24-27, wherein the second molecule is a fluorescent quencher, wherein the fluorescent quencher is selected from FAM. Biotin, digoxin, and dUTP. 29. The visual detection system of any one of embodiments 18-28, wherein the system is present in a lateral flow format.
30. The visual detection system of any one of embodiments 18-28, wherein the target enzyme is a pathogen-derived enzyme, and the system detects the activity of the pathogen-derived enzyme following exposure to an anti-pathogenic agent.
31. The visual detection system of embodiment 30, wherein the pathogen-derived enzyme is a reverse transcriptase enzyme of the Human immunodeficiency virus (HIV), and wherein the anti-pathogenic agent comprises an Antiretroviral therapeutic (ART) agent, optionally, wherein the ART agent is tenofovir-diphosphate (TFV-DP).
32. A lateral flow device/system for detecting/monitoring enzymatic activity of a target enzyme, the device/system comprising a substrate comprising a first end, wherein the first end comprises: (a) a sample loading portion; and (b) a first region comprising: (i) a CRISPR effector system; (ii) a nucleic acid probe comprising a detectable label; and (iii) a first capture region comprising a first binding agent; wherein the CRISPR effector system comprises: at least one CRSPR effector protein having collateral cleavage activity; and at least one sgRNA that directs the at least one CRISPR effector protein to specifically bind to a target nucleic acid sequence, and wherein the target nucleic acid sequence is a sequence generated/synthesized by the enzymatic activity of the target enzyme, wherein the nucleic acid probe is cleavable by collateral cleavage activity of the CRISPR effector system, and wherein when the nucleic acid probe is cleaved a detectable signal is generated from the detectable label or wherein when the nucleic acid probe is cleaved a detectable signal is removed from the detectable label.
33. The lateral flow device of embodiment 32, wherein the sample loading portion further comprises reagents for generation of the target nucleic acid, wherein the reagents comprise: (i) a nucleic acid template; (ii) dNTPs; and (iii) primers.
34. The lateral flow device of embodiment 32 or embodiment 33, wherein the nucleic acid probe comprises a single-stranded RNA or a single-stranded DNA oligonucleotide, and wherein the nucleic acid probe comprises a first molecule attached to the 5′ end and a second molecule attached to the 3′ ends of the oligonucleotide or vice-versa, or optionally wherein the nucleic acid probe comprises a first molecule attached to any base of an oligonucleotide and a second molecule attached to a different base of the oligonucleotide.
35. The lateral flow device of embodiment 33, wherein the first molecule is a fluorescent molecule, and the second molecule is a fluorescent quencher, or vice-versa, wherein the fluorescent molecule is selected from FITC, Cy5, FAM, Texas Red, and Cy7, and wherein the fluorescent quencher is selected from FAM, Biotin, digoxin, and dUTP.
36. The lateral flow device of embodiment 34 or embodiment 35, wherein the first capture region is proximate to and on the same end of the lateral flow substrate as the sample loading portion, wherein the first capture region comprises a first binding agent that specifically binds the first molecule of the nucleic acid probe.
37. The lateral flow device of any one of embodiments 32-36, wherein the device/system further comprises a second capture region, wherein the second capture region is located towards the opposite end of the lateral flow substrate from the first binding region, and wherein the second capture region optionally comprises a second binding agent that specifically binds the second molecule of the nucleic acid probe.
38. The lateral flow device of any one of embodiments 32-37, wherein the sample is a biological sample; wherein the biological sample is optionally a blood, plasma, serum, urine, saliva, mucous, cervicovaginal fluid, lymph fluid, synovial fluid, ascites, pleural effusion, seroma, cerebrospinal fluid, sputum, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.
39. The lateral flow device of embodiment 38, wherein the sample is loaded in the sample loading portion of the substrate, wherein the sample flows from the sample loading portion of the substrate to the first and optionally the second capture regions, wherein when the target enzyme is not active or is absent in the sample, the target nucleic acid sequence is not generated, the CRISPR effector system is inactive, and the nucleic acid probe is not cleaved by the collateral cleavage activity of the at least one CRISPR effector protein to generate a visually detectable signal from the detectable label or to remove a visually detectable signal from the detectable label.
40. The lateral flow device of embodiment 38, wherein the sample is loaded in the sample loading portion of the substrate, wherein the sample flows from the sample loading portion of the substrate to the first and optionally the second capture regions, wherein when the target enzyme is active or present, the target nucleic acid sequence is generated, the CRISPR effector system is active, and the nucleic acid probe is cleaved by collateral cleavage activity of the CRISPR effector protein to generate a visually detectable signal from the detectable label or to remove a visually detectable signal from the detectable label.
41. The lateral flow device of any one of embodiments 32-40, wherein the target enzyme is a pathogen-derived enzyme, and wherein the system detects the activity of the pathogen-derived enzyme activity following exposure to an anti-pathogenic agent.
42. The lateral flow device of embodiment 41, wherein the pathogen-derived enzyme is a reverse transcriptase or a retroviral integrase enzyme of the Human immunodeficiency virus (HIV), and wherein the anti-pathogenic agent is an Antiretroviral therapeutic (ART) agent, and wherein the system is useful in monitoring adherence to ART agent in a subject.
43. A point of care method for monitoring adherence to a therapy targeting a disease-associated enzyme in a subject, the method comprising: (i) obtaining a biological sample from the subject; (ii) contacting the biological sample with (a) a CRISPR effector system comprising: (1) at least one CRISPR effector protein having collateral cleavage activity; and (2) at least one sgRNA that directs the at least one CRISPR effector protein to specifically bind to a target nucleic acid sequence; and (b) a nucleic acid probe comprising a detectable label, wherein the target nucleic acid sequence is a sequence generated/synthesized by the activity of the disease-associated enzyme, and wherein the nucleic acid probe is cleavable by collateral cleavage activity of the at least one CRISPR effector protein, and wherein when the nucleic acid probe is cleaved a detectable signal is generated from the detectable label or wherein when the nucleic acid probe is cleaved a detectable signal is removed from the detectable label; (iii) measuring the detectable signal generated or removed from the detectable label; and (iv) determining a level of adherence to the therapy based on the measured detectable signal.
44. The method of embodiment 43 further comprising adding reagents for generation of the target nucleic acid, wherein the reagents comprise: (i) a nucleic acid template; (ii) dNTPs; and (iii) nucleic acid amplification reagents.
45. The method of embodiment 43 or embodiment 44, wherein the nucleic acid probe comprising the detectable label comprises a first molecule attached to the 5′ end and a second molecule attached to the 3′ ends of an oligo or vice-versa, or optionally wherein the nucleic acid probe comprising the detectable label comprises a first molecule attached to any base of an oligo and a second molecule attached to a different base of the oligo.
46. The method of any one of embodiments 43-45, wherein the nucleic acid probe is an RNA or a DNA oligonucleotide.
47. The method of any one of embodiments 43-46, wherein the nucleic acid probe is a single-stranded RNA or a single-stranded DNA molecule, and wherein the first molecule and the second molecule are covalently attached on the base of the single-stranded RNA or single-stranded DNA molecule.
48. The method of embodiment 47, wherein the first molecule is a fluorescent molecule, and wherein the fluorescent molecule is selected from FITC, Cy5, FAM, Texas Red, and Cy7.
49. The method of embodiment 47 or embodiment 48, wherein the second molecule is a fluorescent quencher, wherein the fluorescent quencher is selected from FAM. Biotin, digoxin, and dUTP.
50. The method of any one of embodiments 43-49, wherein the at least one CRISPR effector protein comprises Cas12a protein.
51. The method of any one of embodiments 43-49, wherein the at least one CRISPR effector protein comprises a Cas protein with similar collateral cleavage activity as Cas12a.
52. The method of embodiment 50 or embodiment 51, wherein the sgRNA is a crRNA molecule.
53. The method of any one of embodiments 43-51, wherein when the disease-associated enzyme is not active or is absent, the target nucleic acid sequence is not generated, the at least one CRISPR effector system is inactive, and the nucleic acid probe is not cleaved by the collateral cleavage activity of the at least one CRISPR effector protein to generate a detectable signal or to remove a detectable signal.
54. The method of any one of embodiments 43-51, wherein when the disease-associated enzyme is active or present, the target nucleic acid sequence is generated, the at least one CRISPR effector system is active, and the nucleic acid probe is cleaved by the collateral cleavage activity of the at least one CRISPR effector protein to generate the detectable signal or to remove a detectable signal.
55. The method of any one of embodiments 43-54, wherein the detectable signal is detected using a detection equipment/instrument.
56. The method of any one of embodiments 43-54, wherein the detectable signal is visually detected by naked eyes without an instrument/detection equipment.
57. The method of any one of embodiments 43-56, wherein the disease-associated enzyme is reverse transcriptase of Human immunodeficiency virus (HIV).
58. The method of any one of embodiments 43-56, wherein the disease-associated enzyme is an integrase of Human immunodeficiency virus (HIV).
59. The method of embodiment 57 or embodiment 58, wherein the therapy comprises an antiretroviral therapy (ART).
60. The method of embodiment 59, wherein the ART comprises tenofovir-diphosphate (TFV-DP).
61. A system for monitoring a drug level in a biological sample, comprising:
   (a) a lateral flow assay device comprising a sample pad, a conjugate pad, a nitrocellulose membrane, and an absorbent pad arranged in fluid communication;
   (b) a CRISPR/Cas detection complex disposed on or within the conjugate pad, wherein the CRISPR/Cas detection complex comprises a guide RNA and a Cas effector enzyme configured to cleave a labeled nucleic acid reporter in response to a drug-responsive nucleic acid signal;
   (c) a reporter configured to produce a detectable signal upon cleavage; and
   (d) a digital reader configured to detect the signal and generate an output indicative of the concentration of the drug in the biological sample.
62. The system of embodiment 61, wherein the Cas effector enzyme is selected from the group consisting of Cas12a (Cpf1), Cas13a, Cas13b, Cas14a, and CasDx1.
63. The system of embodiment 61, wherein the drug-responsive nucleic acid signal comprises absence or low concentrations of the drug level in the biological sample.
64. The system of embodiment 61, wherein the detectable signal is colorimetric, fluorescent, chemiluminescent, or electrochemical.
70. The system of embodiment 61, wherein the reporter comprises a fluorophore-quencher pair, a biotin-fluorescein pair, or a nanoparticle conjugate.
71. The system of embodiment 61, wherein the biological sample is selected from the group consisting of blood, plasma, serum, saliva, and urine.
72. The system of embodiment 61, wherein the digital reader comprises an optical sensor configured to detect reflected light, fluorescence intensity, or luminescence from a test line and a control line on the lateral flow strip.
73. The system of embodiment 61, wherein the digital reader includes a processor configured to normalize and analyze the signal intensity using a calibration curve or algorithm to compute a quantitative drug concentration.
74. A method for determining a drug concentration in a biological sample, comprising:
   (a) introducing the biological sample to a lateral flow assay comprising a CRISPR/Cas detection complex and a labeled reporter;
   (b) activating the CRISPR/Cas detection complex via a drug-responsive nucleic acid signal that correlates with the presence or concentration of the drug;
   (c) detecting a cleavage event of the reporter to generate a measurable signal; and
   (d) processing the measurable signal using a digital reader to determine or estimate the drug concentration and optionally transmitting the result to a remote or local data management platform.
75. A system for integrated drug adherence monitoring comprising:
   (a) a CRISPR-based lateral flow assay configured to detect a drug or metabolite in a biological sample;
   (b) a digital reader configured to quantify the assay signal; and
   (c) a software platform configured to process and store temporal drug level data, compare measured concentrations to expected pharmacokinetic profiles, and provide adherence feedback or dosing recommendations.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from the true scope of the invention.

### Example 1

### REACTR Process

The Cas12a-crRNA complex was created by combining 100 nM LbCas12a (M0653T. New England Biolabs) and 100 nM crRNA (Integrated DNA Technologies) in HIV RT buffer containing 50 mM Tris-HCl (77-86-1. Sigma-Aldrich) at pH 8, 50 mM Potassium Chloride (KCl) (60142-100ML-F. Sigma-Aldrich). 10 mM Magnesium Chloride (MgCl₂) (7786-30-3. Sigma Aldrich), 2 mM dithiothreitol (DTT) (3483-12-2. Sigma-Aldrich), and 0.06% Triton X-100 (9036-19-5. Sigma-Aldrich). The reagents were incubated for 15 minutes at 25° C.

AZT-TP (B1331-007254. BOC Sciences) was diluted in nuclease-free water at varying concentrations from 10⁻⁴ to 10⁻¹⁰ M, 18 μL of a master mix containing RT HIV buffer with 0.2 μL (1 M) MgCl₂ and 0.2 μL (1 M) DTT, 0.2 μL (100 μM) deoxynucleotides (dNTP) (N0447L. New England Biolabs), 0.1 μL (20 μM) RT primer, 0.1 μL (2 μM) RT template, and 1 μL (20 μM) reporters (Integrated DNA Technologies), 10 μL of the Cas12a-crRNA complex, and 10 μL of the drug dilution were combined and added to a black, flat-bottom polystyrene 96-well plate with nonbinding surfaces (3650. Corning). 2 μL of HIV RT enzyme at 20.2 U/μL (LS005009. Worthington Biochemical) in HIV RT buffer was added to the mixture to initiate the reaction. The drug dilutions were compared against no-drug and no-enzyme controls. The positive control replaced the drug dilutions with nuclease-free water while the negative controls replaced the drug dilutions with nuclease-free water and HIV RT with HIV RT buffer. Reactions were incubated at 37° C. in a microplate reader (SpectraMax™ iD3. Molecular Devices) for 90 minutes. Fluorescent measurements were taken every two minutes for the entire incubation period, with one ten-second shake before the first fluorescence measurement.

### Example 2

### Template Design

DNA templates and primer, which were purchased from Integrated DNA Technologies (IDT), were designed to minimize secondary structures and self-dimerization. The DNA templates were designed to have a primer binding region followed by CTAA repeats, the complement crRNA recognition region, the PAM sequence required for Cas12a cleavage, and a 10nt Poly-T tail. A poly-T tail was included to increase fluorescence signal due to crRNA-Cas12a activation from adjacent sequences. The CTAA repeating region ranged from 5 to 35 repeats to reach 80nt and 200nt, respectively. The CTAA repeat was designed to include more Adenosine bases because AZT-TP is a deoxythymidine triphosphate (dTTP) analog and will bind to A's in the template. The final designs of the templates and primer are listed in Table 1.

### Example 3

### Genomic DNA+REACTR

100 μg Human Mixed Genomic DNA (G3041. Promega) was purchased and diluted in nuclease-free water prior to experiments. Genomic DNA was diluted to final concentrations of 4 ng/μL and 10 ng/μL concentrations by adding it to the Master Mix described above, replacing some of the nuclease-free water. The REACTR process was prepared as described above, except stock genomic DNA was added to the master mix as an extra component. Reactions were incubated the same way as described above.

### Example 4

### REACTR+PBMC Lysate

Human PBMCs were purchased from BioIVT and resuspended in 1 mL of M-PER™ Mammalian Protein Extraction Reagent (78501. Thermo Fisher Scientific). The lysed PBMCs were shaken at 250 rotations per minute (rpm) for 10 minutes and were diluted with TE Buffer to 2E6 cells/mL, the concentration of PBMCs in 1 mL of whole blood. PBMC lysate was then diluted with TE buffer to reach final concentrations of 4%, 10%, 25%, and 100% PBMC lysate. 10 μL of AZT-TP spiked in the PBMC lysate was added to the reaction mixture so the final PBMC lysate concentration was ¼^{th} the concentration in the assay. Serial dilutions of clinically relevant levels of AZT-TP with concentrations ranging from 10⁻⁴ to 10⁻¹⁰ M were prepared. The 200nt DNA template was chosen as the template for the master mix, while the rest of the components remained the same. The RT and the Cas complex were prepared the same as described above. Reactions were incubated the same way as described above.

### Example 5

### Data Analysis

The drug-inhibition curves were fit to data from the 30-minute time point. The fluorescence intensities were then normalized using the average of the positive and negative controls. The normalized curves were then fitted to four-parameter logistic regression curves using GraphPad Prism 10 (GraphPad Software).

### Example 6

### REACTR Overview and Process

REACTR measures DNA synthesis by HIV RT using a CRISPR-Cas12a reporter system. A DNA template was engineered such that the complementary DNA (cDNA) synthesized by HIV RT activates the crRNA-Cas12a complex and generates fluorescence through collateral cleavage of a reporter molecule. Non-nucleotide Reverse transcription (RT) inhibitors (NRTIs) and deoxynucleotide triphosphates (dNTPs) compete for incorporation into cDNA (FIG. 1A). At high NRTI concentrations, cDNA chain termination is more favorable, resulting in short DNA fragments that do not contain the crRNA-Cas12a recognition sequence, leaving the Cas12a complexes in the reaction inactivated. Conversely, at low NRTI concentrations, full-length cDNA synthesis is more likely, resulting in long DNA fragments with intact crRNA-Cas12a recognition sequences and high levels of Cas12a complex activation. Thus, the amount of Cas12a activation, as measured through fluorescence, can be used to infer NRTI concentrations (FIG. 1B).

### Example 7

### REACTR Accurately Measures AZT-TP Concentrations in Buffer

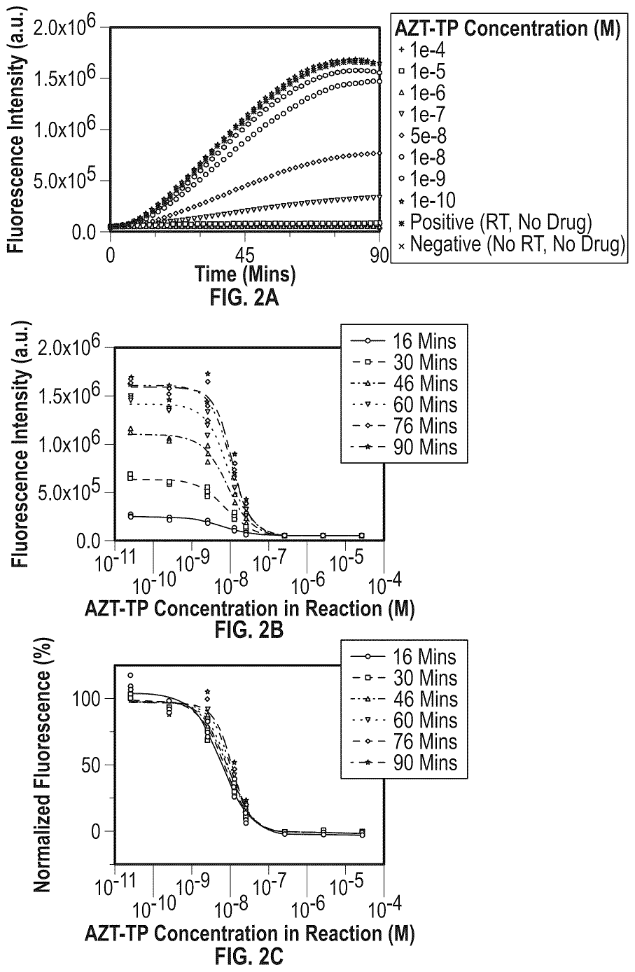

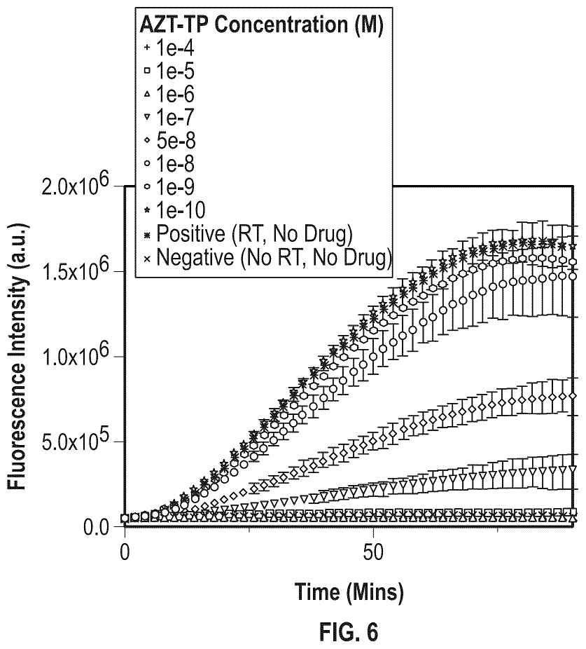

REACTR assay's ability to measure azidothymidine triphosphate (AZT-TP), the active intracellular metabolite of zidovudine—the first ever antiretroviral drug used for HIV treatment and prevention, which is still in use in pediatric treatment regimens, was tested. AZT-TP was spiked into aqueous buffer at concentrations (10⁻⁴ M to 10⁻¹⁰ M) spanning 3 orders of magnitude above and below the physiologically relevant range and these samples were added into REACTR assays with real-time fluorescence measurement for 90 minutes (FIG. 2A, FIG. 6). For reactions containing AZT-TP concentrations >10⁻⁷ M. DNA synthesis was inhibited such that fluorescent curves were comparable to the negative controls (no RT, no drug). At AZT-TP concentrations ≤10⁻⁷ M, fluorescence intensity increased over time, indicating that DNA synthesis by HIV RT activated Cas12a complexes. As expected, higher fluorescence values were obtained at lower AZT-TP concentrations with the lowest drug concentration tested (10⁻¹⁰ M) overlapping with the positive (RT, no drug) control.

A classic sigmoidal enzyme inhibition curve was created for data at 15-minute increments (FIG. 2B); 16, 46, and 76 minutes were chosen to represent 15, 45, and 75 minutes, respectively, since fluorescence data were collected every two minutes. 76 and 90 minutes had overlapping curves because the reaction plateaued around 75 minutes. The longer time points corresponded with a higher fluorescence because there was more time for the activated Cas12a to cleave the reporters.

Next, the inhibition curves were normalized to the corresponding positive and negative controls at each time point to facilitate comparisons across time points (FIG. 2C). Assay incubation time did not have an impact on the normalized enzyme inhibition curves which all overlapped with one another. It was anticipated that the specific time point would not matter because Cas12a cleaves at a consistent rate and a longer time point simply results in more reporters being cleaved. Since all the time points displayed the same normalized fluorescence intensity curve, 30 minutes time point was chosen for the final REACTR Assay since it provides a good tradeoff between maximizing the raw fluorescence signal and providing an assay time that is suitable for rapid point-of-care testing.

### Example 8

### DNA Template Length Impacts REACTR Sensitivity to AZT-TP

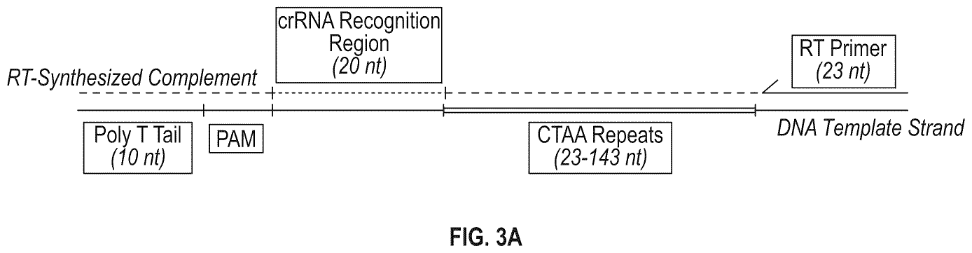

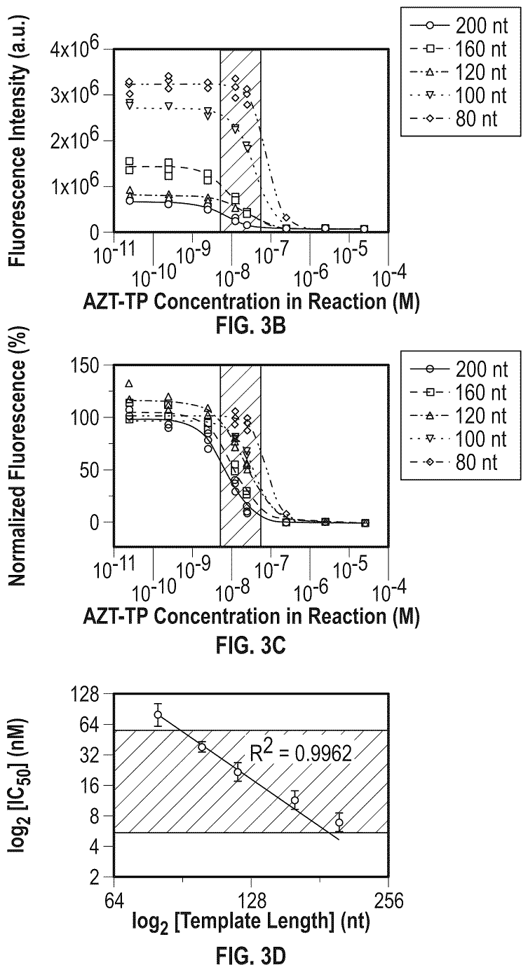

Next, the impact of DNA template length on REACTR's ability to detect AZT-TP was investigated based on findings from work with an intercalating dye reporting system. DNA templates were designed to vary the length of the repeating CTAA region between the RT primer binding region and the crRNA recognition region (FIG. 3A. Table 1). CTAA as the repeating unit was chosen to minimize self-dimerization and secondary structure of the DNA template which can inhibit or slow HIV RT activity. Enriching for adenosine bases also increases the likelihood of AZT-TP binding since the drug is a thymidine analog. Increasing the number of CTAA repeats between the primer and crRNA region provides more opportunities for AZT-TP binding and cDNA chain termination before activation of the crRNA-Cas12a complex.

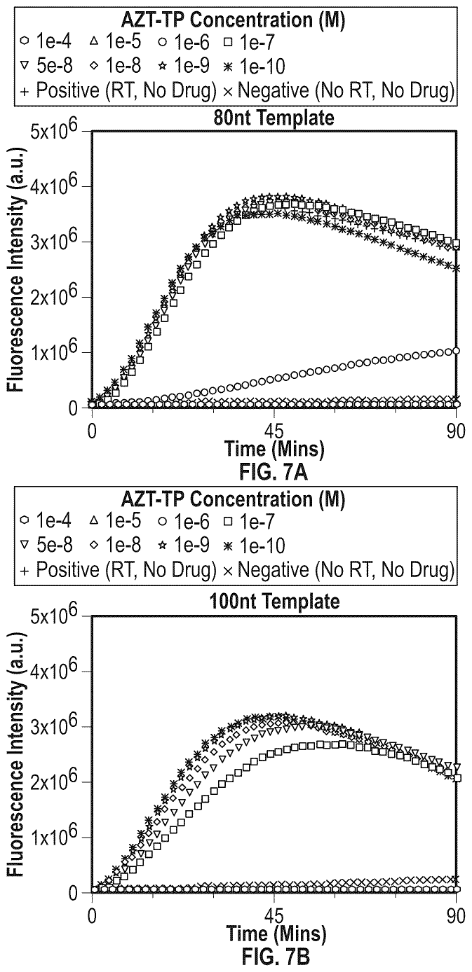

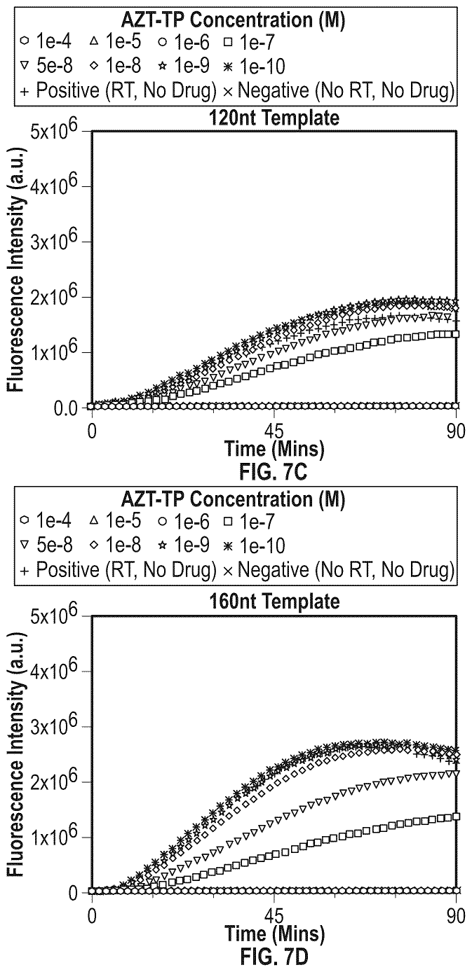

REACTR assay was run with DNA template lengths of 80, 100, 120, 160, and 200 nucleotides (nt) across a range of AZT-TP concentrations (FIG. 3B). A direct relationship between template length and amount of fluorescence generated in the 30 minutes of assay incubation was observed. The shortest template, 80nt, had the highest fluorescence intensity compared to longer templates. This relationship was expected because the same concentration of reagents (dNTP and template) was used for each condition. Thus, dNTP is in greater excess for the shorter template lengths and can more easily incorporate into the cDNA, activating more Cas12a complexes. However, if it is ensured that there are no limiting reagents, it is expected that the steady state for each template will be same. The 80nt template had the greatest fluorescence because it took the fewest bases to reach the crRNA recognition region and activate Cas12a. Thus, a higher proportion of the Cas12a became activated by the 30-minute analysis timepoint. Conversely, the 200nt CTAA template had the lowest overall fluorescence intensity because there were more bases to incorporate before the crRNA recognition region was fully synthesized. Although the 120nt CTAA template deviated from the trend, the rest of the template lengths confirmed that fluorescence intensity increased as template length decreased. Real-time fluorescence intensity curves and inhibition curves for other template lengths are available in FIGS. 7A-7D.

The inhibition curves were normalized to more easily compare effects of the DNA template length on REACTR's sensitivity to AZT-TP in a clinical range of drug concentrations found in PBMCs. After normalization, it was observed that AZT-TP inhibition of REACTR increased with increasing template length, as the inhibition curves shifted to the left (FIG. 3C). This shift occurred because longer templates provided more binding sites for AZT-TP to be incorporated into the complementary DNA (cDNA) strand, increasing the likelihood of cDNA chain termination before reaching the crRNA recognition region. Of note, after normalization, no deviations in the trend for the 120nt template that was seen in FIG. 3B were observed.

This inhibition is also reflected in the calculated IC₅₀ values, which logarithmically decrease as template length increased (FIG. 3D), with a strong linear correlation (R²=0.9962). This linear relationship allows us to design and optimize templates to measure concentrations of AZT-TP that are found in PBMCs of children living with HIV. Moving forward, the 200nt template was selected to allow the most binding regions.

### Example 9

### REACTR Retains Sensitivity in Presence of Genomic DNA

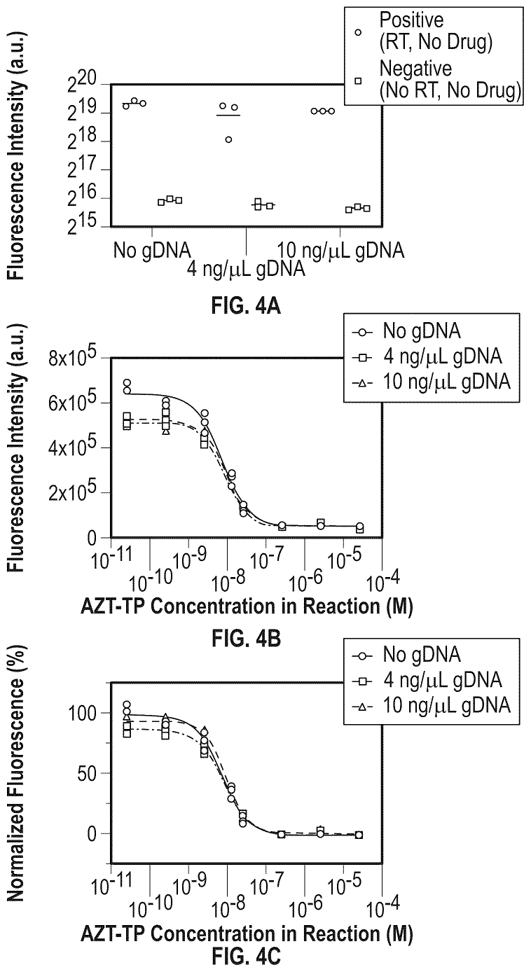

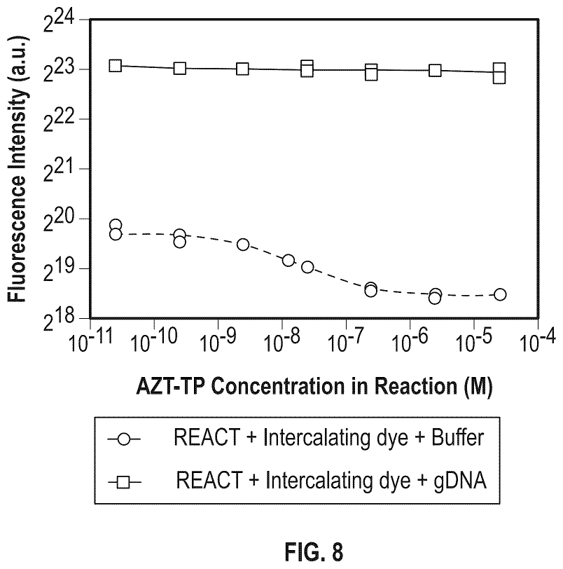

AZT-TP accumulates exclusively in the nuclei of white blood cells; to release and detect AZT-TP, the cells must be lysed, which also releases genomic DNA (gDNA) found in the nucleus. Previous drug inhibition assays used PicoGreen (PG), an intercalating dye, to enable fluorescent readout; however, PG indiscriminately binds to all double-stranded DNA, resulting in an inability to distinguish drug concentrations in the presence of gDNA (FIG. 8). REACTR assay's ability to distinguish between positive and negative controls with gDNA was tested (FIG. 4A). A biologically relevant concentration of gDNA in whole blood at 39.1 ng/μL was selected and diluted in nuclease-free water 1:4 and 1:10 to achieve final concentrations of 10 ng/μL and 4 ng/μL, respectively. The dilution factors were chosen to incorporate theoretical sample dilutions that have been previously demonstrated with other enzymatic assays. The addition of gDNA had no impact on the REACTR assay indicating that REACTR is highly sensitive and specific to the DNA strand of interest.

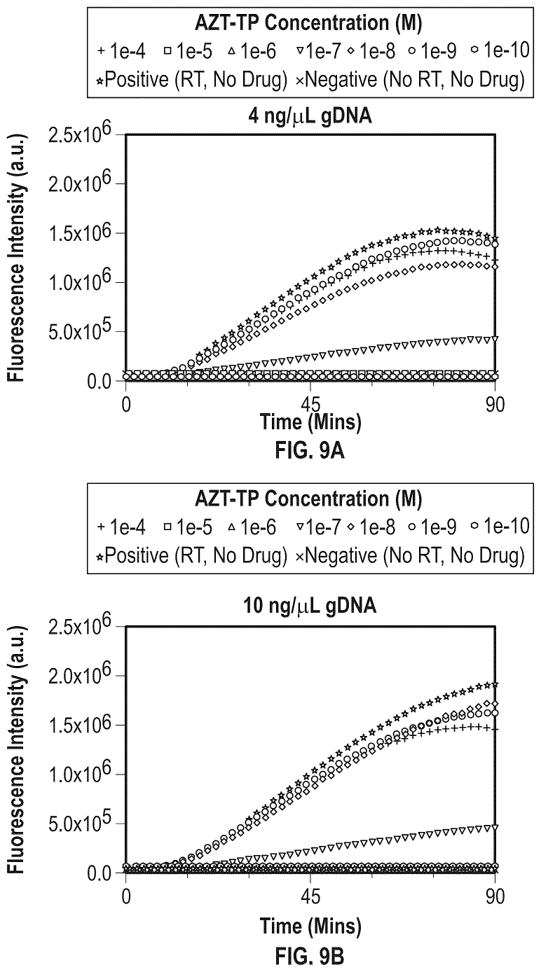

Next. REACTR assay's sensitivity in differentiating various AZT-TP concentrations in the presence of different concentrations of gDNA (FIGS. 9A-9B) was tested. The raw drug inhibition curves showed slightly higher fluorescence in the gDNA conditions, possibly due to light scattering from the gDNA. After normalization, no difference between 10 ng/μL and 4 ng/μL gDNA concentrations compared to the condition without gDNA, were observed thereby demonstrating that the REACTR assay can successfully detect AZT-TP with the same sensitivity across various gDNA concentrations (FIGS. 4B-4C).

### Example 10

### REACTR is Uninhibited by PBMC Lysate

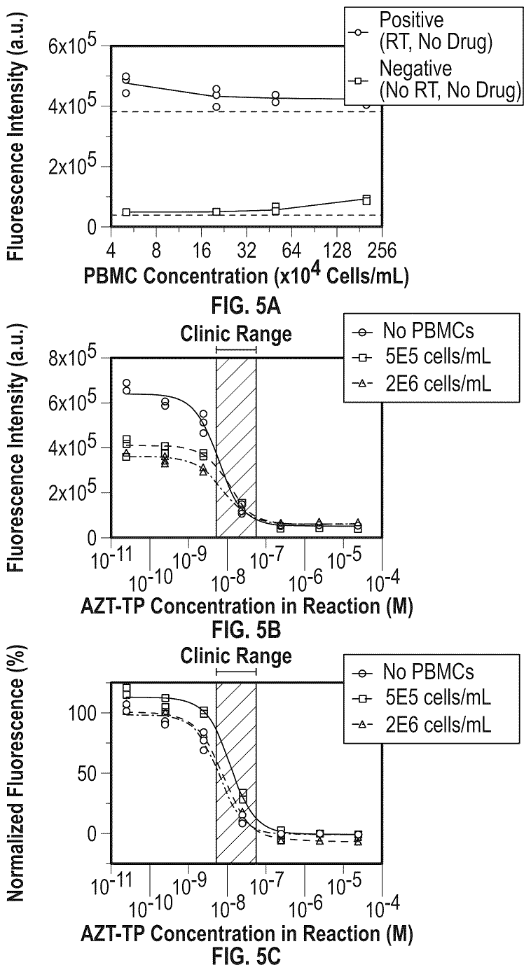

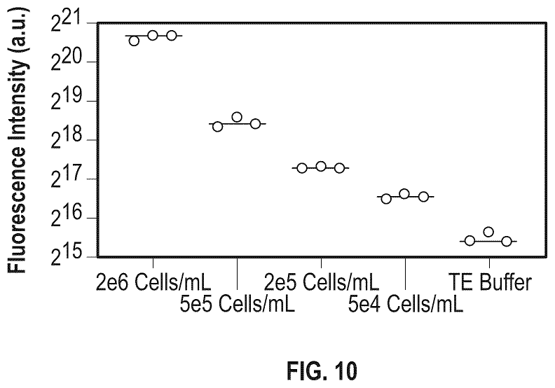

Given that AZT-TP accumulates in the nuclei of PBMCs and must be lysed to access AZT-TP, the influence of PBMC lysate on the REACTR assay was investigated next. PBMCs at a biologically relevant concentration (2E6 cells/mL) were lysed and selected dilution ratios to reach specific PBMC concentrations: 4%, 10%, 25%, and 100%. Sufficient lysis of PBMCs was confirmed using PG fluorescence (FIG. 10) and the impact of this lysate on REACTR positive and negative controls, was evaluated (FIG. 5A). Comparable fluorescence compared to the controls was observed. All the conditions with PBMC lysate had higher fluorescence levels than the controls, which could potentially be due to light scattering from the PBMC lysate. To ensure the concentration of AZT-TP will be detectable in clinical samples, 100% and 25% dilutions were selected going forward.

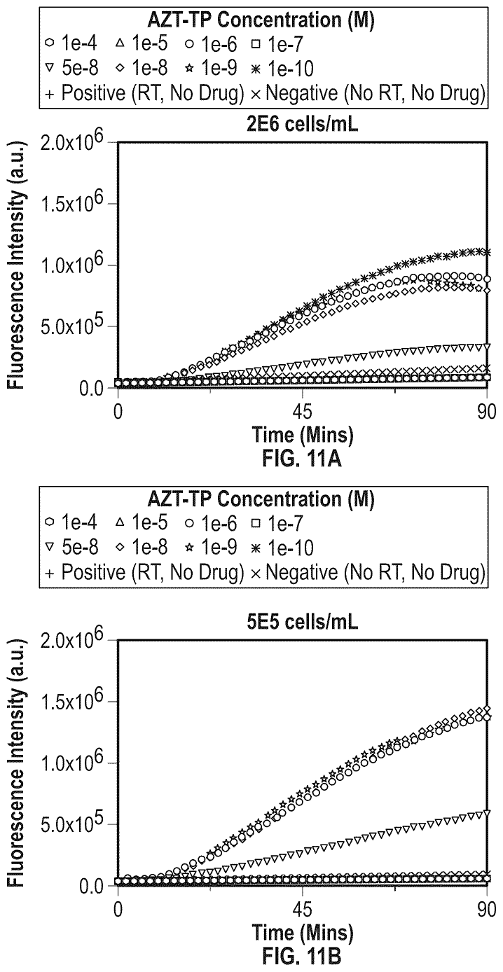

After the PBMC dilutions were selected. AZT-TP was spiked with the PBMC lysate to examine how the drug inhibition curve changed (FIG. 5B). PBMC lysate concentrations of 100% and 25% were compared with a no PBMC lysate control. Both selected PBMC concentrations showed a smaller inhibition range than the no-PBMC condition, indicating that PBMC lysate did not compromise the drug inhibition curve. Interestingly, normalized inhibition fluorescence curves, compared to positive and negative controls, were similar (FIG. 5C). While the 25% condition slightly differed from the control and 100%, the sinusoidal shape was consistent, only shifted upwards. Normalization eliminated differences in the AZT-TP inhibition range (FIG. 5C), confirming that PBMC lysate did not compromise the REACTR assay's integrity. Real-time fluorescence intensity curves at different PBMC lysate concentrations are available in FIGS. 11A and 11B. Given these promising results, future work will use clinical PBMC samples from patients taking AZT.

### Example 11

### REACTR LFA can Detect Clinically Relevant TFV-DP Levels

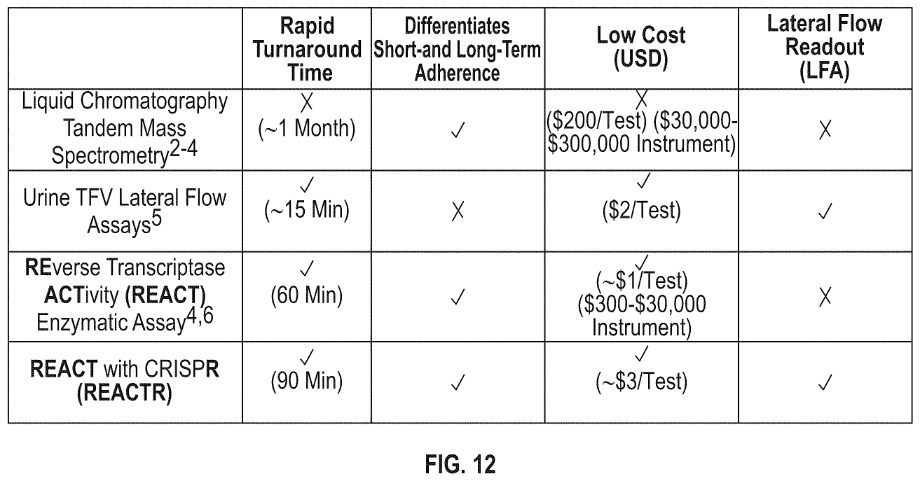

Although drug concentration feedback provides objective information that may help to improve HIV medication adherence and health outcomes, conventional testing with liquid chromatography tandem mass spectrometry (LC-MS/MS) is arduous and expensive (FIG. 12). Urine tenofovir lateral flow assays (LFAs) for measuring adherence are rapid and inexpensive but cannot distinguish between recent (<1 week) and long-term (1-3 month) adherence. The REverse transcriptase ACTivity (REACT) assay rapidly measures tenofovir diphosphate (TFV-DP), a metabolite that indicates long-term oral PrEP/ART adherence, based on the drug's inhibition of DNA synthesis by HIV reverse transcriptase (RT) enzyme.

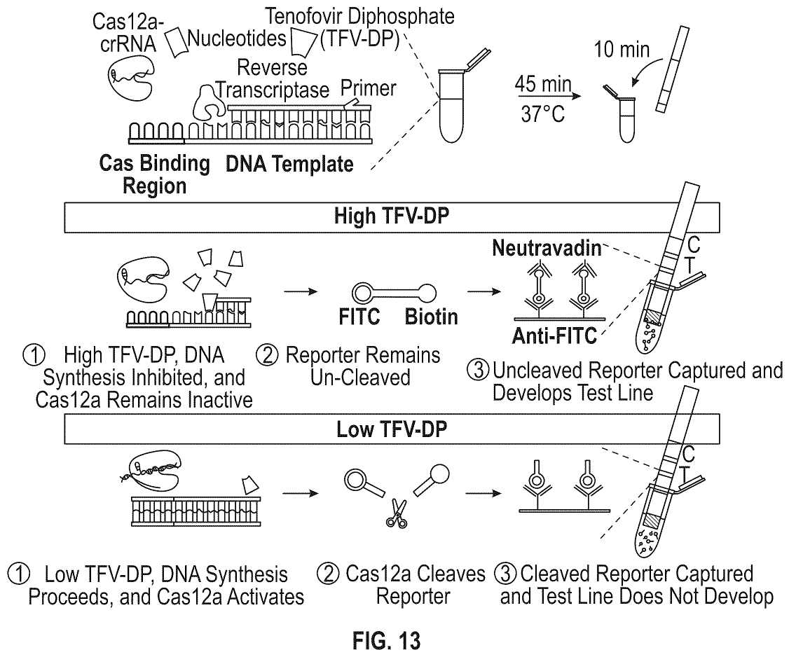

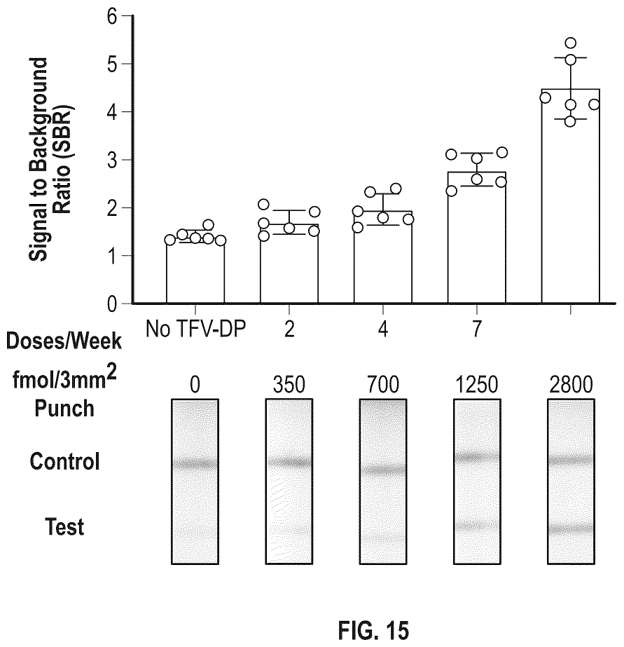

CRISPR RNA, Cas12a. DNA template, nucleotides. HIV-RT, biotin-FITC reporters were combined, and spiked with TFV-DP concentrations corresponding to 2, 4, and 7 doses/week (350, 700, and 1250 fmol/3 mm² punch) into rehydrated dried blood spots from three donors not receiving any antiretroviral drugs (FIG. 15). The test line of the LFA strip detects the presence of uncleaved CRISPR reporters, so a “no drug” and “no enzyme” conditions were also included to determine the minimum and maximum signal achievable. We incubated REACT assays at 37° C. for 45 minutes and visualized reaction products on commercially available LFA strips (Abingdon Health PCRD FLEX™) that were immersed in reaction tubes for 10 minutes. We scanned the LFAs to quantify the test line intensity, calculated signal-to-background ratios (SBR), and determined a positivity threshold at 3 standard deviations above the mean of the “no drug” condition to allow quantitative analysis of our results. Reagent concentrations were optimized (Cas enzyme, nucleotides, and reaction time) to tune the LFA signal to be above the positive threshold (FIG. 13).

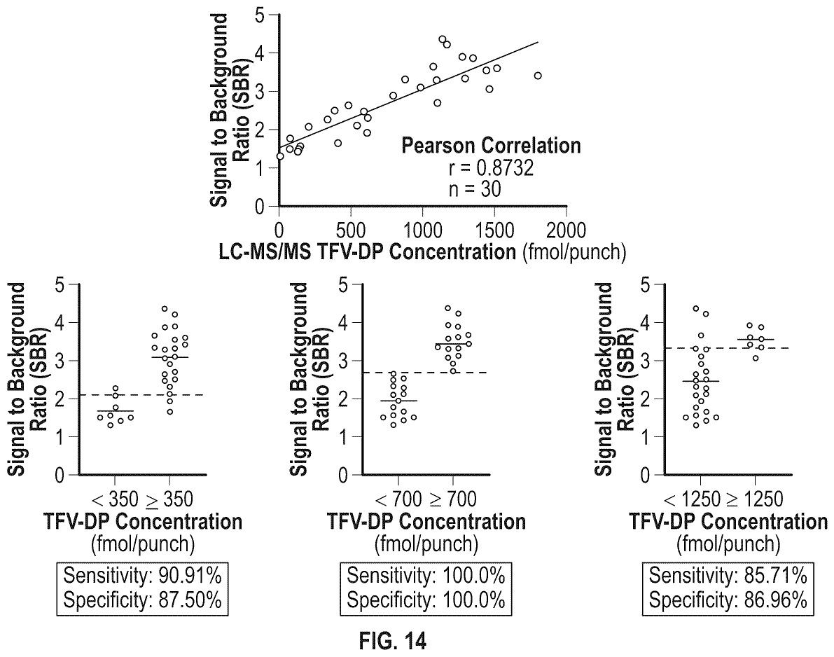

No-enzyme controls had an average SBR of 4.64±0.39 and a coefficient of variation of 8.37% which indicates low inter-strip variability. SBR values correlated with LC-MS TFV-DP concentrations for DBS samples (r=0.8732, n=30) (FIG. 14). The assay had high sensitivity (100.0%) and specificity (100.0%) in distinguishing samples above the 700 fmol/punch TFV-DP threshold (SBR=2.675) for adequate PrEP adherence (≥4 doses/week) (FIG. 14).

TABLE 1
DNA Sequences For the Template Design

| Names | Sequence |
|---|---|
| 80 nt | TTTTTTTTTTTTTGATGATGTGAAGGTGTTGTCGCTAAC |
| CTAA | TAACTAACTAACTAACTAACTAACTAACTAACTAACTAC |
| Template | TATCTTTCCTCTTAATTCGACG |
| 100 nt | TTTTTTTTTTTTTGATGATGTGAAGGTGTTGTCGCTAAC |
| CTAA | TAACTAACTAACTAACTAACTAACTAACTAACTAACTAA |
| Template | CTAACTAACTAACTAACTACTATCTTTCCTCTTAATTCG |
| | ACG |
| 120 nt | TTTTTTTTTTTTTGATGATGTGAAGGTGTTGTCGCTAAC |
| CTAA | TAACTAACTAACTAACTAACTAACTAACTAACTAACTAA |
| Template | CTAACTAACTAACTAACTAACTAACTAACTAACTAACTA |
| | CTATCTTTCCTCTTAATTCGACG |
| 160 nt | TTTTTTTTTTTTTGATGATGTGAAGGTGTTGTCGCTAAC |
| CTAA | TAACTAACTAACTAACTAACTAACTAACTAACTAACTAA |
| Template | CTAACTAACTAACTAACTAACTAACTAACTAACTAACTA |
| | ACTAACTAACTAACTAACTACTATCTTTCCTCTTAATTC |
| | GACG |
| 200 nt | TTTTTTTTTTTTTGATGATGTGAAGGTGTTGTCGCTAAC |
| CTAA | TAACTAACTAACTAACTAACTAACTAACTAACTAACTAA |
| Template | CTAACTAACTAACTAACTAACTAACTAACTAACTAACTA |
| | ACTAACTAACTAACTAACTAACTAACTAACTAACTAACT |
| | AACTAACTAACTAACTAACTACTATCTTTCCTCTTAATT |
| | CGACG |
| DNA | CGTCGAATTAAGAGGAAAGATAG |
| Primer | |
| crRNA | TACTACACTTCCACAACAGC |
| Recog- | |
| nition | |
| Region | |
| Fluor- | /56-FAM/TTATT/3IABKFQ/ |
| escence | |
| Reporter | |
| gRNA | rUrArA rUrUrU rCrUrA rCrUrA rArGrU |
| | rGrUrA rGrArU rArUrG rArUrG rUrGrA |
| | rArGrG rUrGrU rUrGrU rCrG |

While illustrative embodiments have been illustrated and described, it will be appreciated that various changes can be made therein without departing from the spirit and scope of the invention.

1. A system for detecting a target enzyme, the system comprising:
(A) a CRISPR effector system comprising:
(i) at least one CRISPR effector protein having collateral cleavage activity; and
(ii) at least one sgRNA that directs the at least one CRISPR effector protein to specifically bind to a target nucleic acid sequence; and
(B) a detection system comprising a nucleic acid probe comprising a detectable label,
wherein the target nucleic acid sequence is a sequence generated/synthesized by the activity of the target enzyme, and wherein the nucleic acid probe is cleavable by collateral cleavage activity of the at least one CRISPR effector protein, and wherein when the nucleic acid probe is cleaved a detectable signal is generated from the detectable label or wherein when the nucleic acid probe is cleaved a detectable signal is removed from the detectable label.

2. The system of claim 1, wherein the system further comprises reagents for generation of the target nucleic acid, and wherein:
(a) the reagents comprise: (i) a nucleic acid template; (ii) dNTPs; and (iii) primers; and
(b) the at least one CRISPR effector protein comprises one or more thermostable Cas proteins possessing collateral activity, and wherein the one or more Cas protein is a Type V Cas.

3. The system of claim 1, wherein the at least one CRISPR effector protein comprises a Cas 12a protein or a protein with similar bypass/collateral cleavage activity as Cas12a.

4. The system of claim 1, wherein the at least one sgRNA is a crRNA molecule.

5. The system of claim 1, wherein
(i) when the target enzyme is not active or is absent, the target nucleic acid sequence is not generated, the CRISPR effector system is inactive, and the nucleic acid probe is not cleaved by the collateral cleavage activity of the at least one CRISPR effector protein to generate the detectable signal from the detectable label or to remove the detectable signal from the detectable label; and
(ii) when the target enzyme is active or present, the target nucleic acid sequence is generated, the CRISPR effector system is active, and the nucleic acid probe is cleaved by the collateral cleavage activity of the at least one CRISPR effector protein to generate the detectable signal or to remove the detectable signal from the detectable label.

6. The system of claim 1, wherein the generation or removal of the detectable signal is detected by:
(i) using a detection equipment/instrument; or
(ii) naked eyes without an instrument/detection equipment.

7. The system of claim 1, wherein the nucleic acid probe is an RNA or DNA oligonucleotide, and wherein the nucleic acid probe comprises:
(i) a first molecule attached to the 5′ end and a second molecule attached to the 3′ ends of the oligonucleotide or vice-versa; or
(ii) a first molecule attached on any base or multiple bases of the oligonucleotide and a second molecule attached to a different base of the oligonucleotide.

8. The system of claim 7, wherein the nucleic acid probe is a single-stranded RNA or a single-stranded DNA oligonucleotide, and wherein the first molecule and the second molecule are covalently attached on the base of the single-stranded RNA or single-stranded DNA oligonucleotide.

9. The system of claim 7, wherein:
(i) the first molecule is a fluorescent molecule selected from FITC, Cy5, FAM, Texas Red, and Cy7; and
(ii) the second molecule is a fluorescent quencher, wherein the fluorescent quencher is selected from BHQ-1, BHQ-2, Dabcyl, and Iowa Black FQ.

10. The system of claim 1, wherein the system is present in a lateral flow format.

11. The system of claim 1, wherein the target enzyme is a pathogen-derived enzyme, and wherein the system detects the activity of the pathogen-derived enzyme following exposure to an anti-pathogenic agent.

12. The system of claim 11, wherein the pathogen-derived enzyme is a reverse transcriptase enzyme or a retroviral integrase of the Human immunodeficiency virus (HIV), and wherein the anti-pathogenic agent is an Antiretroviral therapeutic (ART) agent.

13. A lateral flow device/system for detecting/monitoring enzymatic activity of a target enzyme, the device/system comprising a substrate comprising a first end, wherein the first end comprises:
(a) a sample loading portion; and
(b) a first region comprising:
(i) a CRISPR effector system;
(ii) a nucleic acid probe comprising a detectable label; and
(iii) a first capture region comprising a first binding agent;
wherein the CRISPR effector system comprises:
at least one CRSPR effector protein having collateral cleavage activity; and
at least one sgRNA that directs the at least one CRISPR effector protein to specifically bind to a target nucleic acid sequence, and
wherein the target nucleic acid sequence is a sequence generated/synthesized by the enzymatic activity of the target enzyme, wherein the nucleic acid probe is cleavable by collateral cleavage activity of the CRISPR effector system, and wherein when the nucleic acid probe is cleaved a detectable signal is generated from the detectable label or wherein when the nucleic acid probe is cleaved a detectable signal is removed from the detectable label.

14. The lateral flow device of claim 13, wherein the sample loading portion further comprises reagents for generation of the target nucleic acid, wherein the reagents comprise: (i) a nucleic acid template; (ii) dNTPs; and (iii) primers.

15. The lateral flow device of claim 13, wherein the nucleic acid probe comprises a single-stranded RNA or a single-stranded DNA oligonucleotide, and wherein the detectable label comprises:
(i) a first molecule attached to the 5′ end and a second molecule attached to the 3′ ends of the oligonucleotide or vice-versa; or
(ii) a first molecule attached to any base of an oligonucleotide and a second molecule attached to a different base of the oligonucleotide.

16. The lateral flow device of claim 15, wherein the first capture region is proximate to and on the same end of the lateral flow substrate as the sample loading portion, and wherein the first capture region comprises a first binding agent that specifically binds the first molecule of the nucleic acid probe.

17. The lateral flow device of claim 15, wherein the device/system further comprises a second capture region, wherein the second capture region is located towards the opposite end of the lateral flow substrate from the first binding region, and wherein the second capture region optionally comprises a second binding agent that specifically binds the second molecule of the nucleic acid probe.

18. The lateral flow device of claim 13, wherein the sample is a biological sample; wherein the biological sample is selected from a blood, plasma, serum, urine, saliva, mucous, cervicovaginal fluid, lymph fluid, synovial fluid, ascites, pleural effusion, seroma, cerebrospinal fluid, sputum, aqueous or vitreous humor, any bodily secretion, a transudate, an exudate (for example, fluid obtained from an abscess or any other site of infection or inflammation), fluid obtained from a joint (for example, a normal joint or a joint affected by disease, such as rheumatoid arthritis, osteoarthritis, gout or septic arthritis), and a swab of skin or mucosal membrane surface.

19. The lateral flow device of claim 18, wherein the sample is loaded in the sample loading portion of the substrate, wherein the sample flows from the sample loading portion of the substrate to the first and optionally the second capture regions, wherein:
(i) when the target enzyme is not active or is absent in the sample, the target nucleic acid sequence is not generated, the CRISPR effector system is inactive, and the nucleic acid probe is not cleaved by the collateral cleavage activity of the at least one CRISPR effector protein to generate a visually detectable signal from the detectable label or to remove a visually detectable signal from the detectable label; and
(ii) when the target enzyme is active or present, the target nucleic acid sequence is generated, the CRISPR effector system is active, and the nucleic acid probe is cleaved by collateral cleavage activity of the CRISPR effector protein to generate a visually detectable signal from the detectable label or to remove a visually detectable signal from the detectable label.

20. The lateral flow device of claim 18, wherein the target enzyme is a pathogen-derived enzyme, wherein the device detects the activity of the pathogen-derived enzyme following exposure to an anti-pathogenic agent targeting the pathogen-derived enzyme.

21. The lateral flow device of claim 20, further comprising a digital reader configured to detect the visually detectable signal from the detectable label and generate an output indicative of the enzymatic activity of the target enzyme and correlate it with a concentration/level of the anti-pathogenic agent in the biological sample.

22. The lateral flow device of claim 21, wherein the digital reader comprises:
(i) an optical sensor configured to detect the visually detectable signal from the detectable label; and
(ii) a processor configured to normalize and analyze the signal intensity using a calibration curve or algorithm to compute a quantitative anti-pathogenic agent concentration.

23. A point of care method for monitoring adherence to a therapy targeting a disease-associated enzyme in a subject, the method comprising:
(i) obtaining a biological sample from the subject;
(ii) contacting the biological sample with
(a) a CRISPR effector system comprising:
(1) at least one CRISPR effector protein having collateral cleavage activity; and
(2) at least one sgRNA that directs the at least one CRISPR effector protein to specifically bind to a target nucleic acid sequence; and
(b) a nucleic acid probe comprising a detectable label,
wherein the target nucleic acid sequence is a sequence generated/synthesized by the activity of the disease-associated enzyme, and wherein the nucleic acid probe is cleavable by collateral cleavage activity of the at least one CRISPR effector protein, and wherein when the nucleic acid probe is cleaved a detectable signal is generated from the detectable label or wherein when the nucleic acid probe is cleaved a detectable signal is removed from the detectable label;
(iii) measuring the detectable signal generated or removed from the detectable label; and
(iv) determining a level of adherence to the therapy based on the measured detectable signal.

24. The method of claim 23 further comprising adding reagents for generation of the target nucleic acid, wherein the reagents comprise: (i) a nucleic acid template; (ii) dNTPs; and (iii) primers.
